(19) United States
(12) Patent Application Publication
(10) Pub. No.: US 2024/0383959 A1
(43) Pub. Date: Nov. 21, 2024

(54) CELL-FREE METHODS AND COMPOSITIONS COMPRISING TAGLESS THERAPEUTIC HORMONES

(71) Applicant: Northwestern University, Evanston, IL (US)

(72) Inventors: Ariel Nicole Thames, Evanston, IL (US); Madison Ann DeWinter, Evanston, IL (US); Weston Kevin Kightlinger, Evanston, IL (US); Michael Christopher Jewett, Evanston, IL (US)

(21) Appl. No.: 18/690,687

(22) PCT Filed: Sep. 9, 2022
(86) PCT No.: PCT/US2022/076180

§ 371 Date: Mar. 8, 2024

Related U.S. Application Data
(60) Provisional application No. 63/261,045, filed on Sep. 9, 2021.

(51) Int. Cl. C07K 14/575; C07K 14/605; C07K 14/61; C07K 14/62; C07K 14/635; C07K 14/65; C07K 14/655

(52) U.S. Cl. CPC C07K 14/575; C07K 14/605; C07K 14/61; C07K 14/62; C07K 14/635; C07K 14/65; C07K 14/655; C07K 2319/22; C07K 2319/35

(57) ABSTRACT

The present disclosure relates to stable, cost-effective, cell-free systems, compositions, methods, and kits for bio-manufacturing and purifying therapeutic hormones. The systems, methods, and kits allow for cell-free bio-manufacturing of the desired hormone products in cell-free conditions, wherein the system can be stably stored or transported, in lyophilized form.

### CROSS-REFERENCE TO RELATED APPLICATIONS

This application claims priority to U.S. Provisional Application 63/261,045, filed Sep. 9, 2021, the content of which is incorporated herein by reference in its entirety.

### STATEMENT REGARDING FEDERALLY SPONSORED RESEARCH OR DEVELOPMENT

This invention was made with government support under CBET-1936789 awarded by the National Science Foundation and HDTRA1-15-10052/P00001 awarded by the Defense Thread Reduction Agency. The government has certain rights in the invention.

### REFERENCE TO AN ELECTRONIC SEQUENCE LISTING

The contents of the electronic sequence listing (702581.02196.xml; Size: 30,791 bytes; and Date of Creation: Aug. 26, 2022) is herein incorporated by reference in its entirety.

### FIELD

The present technology relates to cell-free systems, methods, and kits for bio-manufacturing a desired product, such as a protein, from readily available and inexpensive components. Particularly disclosed herein are systems, methods, and kits providing a thermostable, low-cost, cell-free protein synthesis platform for the production and purification of therapeutic hormones.

### BACKGROUND

Protein hormones can be used to treat a wide variety of chronic diseases, including diabetes, obesity, and osteoporosis. Yet access to the vital medicines remains difficult in resource-limited settings. One of the primary challenges to distributing these medicines is centralized manufacturing and the requirement of cold-chain distribution.

### SUMMARY

The present disclosure relates to methods, systems, kits, and compositions comprising a cell-free protein synthesis reaction to produce therapeutic peptides, such as therapeutic hormones. The present technology can be used to fill an unmet need by providing a low-cost manufacturing platform that is stable outside of the cold chain to distribute and manufacture proteins, for example, therapeutic hormones.

Disclosed herein is an expression and purification strategy for a panel of therapeutically relevant protein hormones that enables (i) point-of-care production, (ii) plug-and-play on-demand synthesis, and (iii) an on-site purification scheme that yields a protein hormone with an identical sequence to the endogenous hormone.

### BRIEF DESCRIPTION OF THE DRAWINGS

FIG. 1a-1b. (a) Lyophilized cell-free expression enables point-of-care peptide hormone manufacturing, purification, and patient delivery. A proposed scheme for point-of-care manufacturing where centrally manufactured CFPS reactions are lyophilized for transportation and storage at room temperature. When needed, the lyophilized CFPS pellets can be rehydrated with plasmid encoding the desired peptide hormone. After the CFPS reaction is complete, the peptide hormones can be purified to eliminate background lysate material and delivered to patients. (b) Exemplary DNA design of Hormone-Protein Fusions. In this example, hormone-protein fusion genetic constructs were designed with a CAT enhancer (MEKKI amino acid sequence, SEQ ID NO: 1) (pink) at the 5′ end to normalize cell-free expression. A purification tag (orange) was joined to a GSSG amino acid sequence linker (green), which allows flexibility, for example, for tag-bead/resin interactions. Not all constructs necessarily require a linker. An internal fusion protein (cyan) intervenes between the tag+linker and the protein of interest (gray). The internal fusion protein imparts stability and solubility on the protein of interest when expressed in cell-free reactions, and can also be cleavable (e.g., SUMO), enabling separation of the protein of interest from other components during purification. Ultimately this yields stable and soluble cell-free expression and purification of tagless protein.

FIG. 2a-2c. Fusion with a SUMO protein allows successful expression of peptide hormones in both standard and lyophilized CFPS reactions. (a) Quantification of StrepII-SUMO-hormone production by radioactive 14C-leucine incorporation is represented for both standard (dark grey) and lyophilized (white) CFPS reactions. All samples (n=3) are shown with error bars representing 1 s.d. (b) An autoradiogram of an SDS-PAGE gel for a mixture of the replicates from panel (a) is shown. Purple arrows point to expected masses. (c) Shows the quantification of additional SUMO tagged hormones produced with standard or lyophilized CFPS reactions. 15 μl reactions were lyophilized for at least 5 hours with a condenser set to −80° C. and a vacuum pressure of 100 mTorr. Cell-free expression of SUMO tagged hormones was tested by incorporation of 14C-Leucine. Total and soluble expression of peptide hormones with sfGFP as a positive control is shown. All constructs express at a level comparable to sfGFP. Lyophilization does not decrease expression. All constructs are very soluble. Error bars represent standard deviations of triplicate CFPS reactions.

FIG. 3a-3d. A simultaneous 2-in-1 enzymatic purification scheme isolates unmodified peptide hormones. (a) The scheme for 2-in-1 purification is shown. (b) A Coomassie stained SDS-PAGE gel shows intact, tagless peptide hormones after purification. The band in each lane corresponding to the full length, tagless hormone is highlighted with a purple arrow. Densitometry was used to obtain the estimated purity. (c) A BCA assay was used to quantify the total protein recovery after purification (n=2) and the hormone recovery was calculated based on the purity by densitometry in panel (b) (white). The recovery was calculated by comparing to the production measured by radioactive leucine incorporation before purification (dark grey) (n=3). Error bars represent 1 s.d. (d) A table showing the typical initial dose for each of these medications and the CFPS reaction volume necessary to produce the dose assuming the same yields from panel c are maintained during scale up.

FIG. 4a-4c. Characterization of GH by ELISA, LC-MS, AlphaLISA, and BLI, shows cell-free produced growth hormone is intact and capable of binding its receptor. (a) GH identification and quantification was determined by sandwich ELISA with 4 dilutions and intact protein LC-MS. The observed mass (22129.34 Da) is shown on a deconvoluted spectrum, and it matches the expected average reduced mass of GH (theoretical: 22129.05 Da) with an error of 13 ppm. (b) GH and GH receptor concentrations were cross titrated, and binding was assessed with AlphaLISA. (c) GH binding to the extracellular domain of its receptor was assessed by BLI. Kinetic curve fitting was unable to be performed due to lack of observed dissociation.

FIG. 5a-5b. SUMO fusions improve molar expression over untagged hormones. (a) A panel of tagless peptide hormones (purple arrows) was screened for expression in crude BL21 extract. Incorporation of Fluorotect, a reagent with fluorescently labeled lysine residues, into the cell-free produced hormones was detected by fluorescent imaging of an SDS-PAGE gel. (b) Fluorotect incorporation was similarly used to screen expression of SUMO-hormone fusions (purple arrows) in crude BL21 extract. More full-length constructs were detected when the hormones were fused with SUMO.

FIG. 6a-6e. Screen of a panel of peptide hormones in lysates from different E. coli strains for improved expression. (a) A panel of peptide hormones was screened for expression in BL21. The same figure from FIG. 5b is repeated here for comparison. Incorporation of Fluorotect, a reagent with fluorescently labeled lysine residues, into the cell-free produced hormones was detected by fluorescent imaging of an SDS-PAGE gel. Lanes are labeled with sample numbers given in panel (e). Fluorotect incorporation was similarly used to screen expression in PUREfrex 2.1 (b), Clm24 (c), and 759 (d) extracts. (e) A summary of expression systems which produced full length peptide hormones is shown. Green checks indicate full length expression and red crosses indicate that full length expression was not observed.

FIG. 7. Screen of the purification procedure on the full peptide hormone panel. After expression in 759 extract and undergoing the full purification procedure, an initial screen showed recovery of untagged GLP-1 mut, GLP-1, GH, and PTH (purple arrows) on a Coomassie stained SDS-PAGE gel.

FIG. 8a-8b. Quantitative determination of the improvement of SUMO fusion peptide hormone expression over untagged hormones. (a) Incorporation of radioactive 1+C-leucine was used to quantify protein expression of untagged (black), CAT-enhancer sequence tagged (purple), and CAT-enhancer SUMO fusion (orange) hormones produced in CFPS. Darker shades represent total hormone expression and lighter shades represent soluble expression. GFP was included as a well-expressing, soluble positive control for comparison. N=3 replicates were performed for each sample, and error bars represent 1 s.d. (b) An autoradiogram of an SDS-PAGE gel of a mixture of the same samples from (a) shows that full length constructs are expressed (purple arrows). Hormones with a CAT-enhancer SUMO fusion are shown on the left, with a CAT-enhancer only in the middle, and without any tags on the right.

FIG. 9a-9f. Characterization of GLP-1 and GLP-1 mut by LC-MS, AlphaLISA, and BLI indicates cell-free produced hormone is intact and may be binding to its receptor. (a) Peptide liquid chromatography-mass spectrometry (LC-MS) data shows intact GLP-1 mut is recovered after purification. The full scan is shown with insets displaying zooms on the isotopic distributions of the +5 charge state (703.54 m/z) and the +4 charge state (879.18 m/z). The monoisotopic masses observed (3510.67 Da) match the theoretical monoisotopic mass of GLP-1 mut (3510.72 Da) with an error of 15 ppm. (b) Peptide LC-MS data shows intact glucagon-like peptide (GLP-1) is recovered after purification. The full scan is shown with insets displaying zooms on the isotopic distributions of the +5 charge state (671.963 m/z) and the +4 charge state (839.66 m/z). The monoisotopic masses observed (3353.62 Da) match the theoretical monoisotopic mass of GLP-1 (3353.67 Da) with an error of 15 ppm. MS/MS analysis of the ions in the +5 charge state confirms the expected sequence of GLP-1 mut (c) and GLP-1 (d), respectively. The b and y ions that were detected are indicated, with purple representing the +1 charge state, blue representing the +2 charge state, and orange representing the +3 charge state of the fragments. If fragments were detected in multiple charge states, the lowest charge state is represented. (e) GLP-1 and GLP-1 receptor concentrations were cross titrated, and binding was assessed with AlphaLISA. (f) GLP-1 binding to the extracellular domain of its receptor was assessed by BLI. No binding was observed with GLP-1 produced in CFPS or the commercially available GLP-1, likely because the hormone is too small to detect on the instrument.

FIG. 10a-10c. Characterization of PTH by ELISA, LC-MS, AlphaLISA, and BLI, indicates hormone is intact and shows some binding with its receptor. (a) PTH identification and quantification was determined by sandwich ELISA with 4 dilutions and intact protein LC-MS. The observed mass (9424.66 Da) on a deconvoluted spectrum matches the expected average mass of PTH (theoretical: 9424.73 Da) with an error of 7.4 ppm. (b) PTH and PTH receptor concentrations were cross titrated, and binding was assessed with AlphaLISA. (c) PTH binding to the extracellular domain of its receptor was assessed by BLI. A large background signal for both commercially produced PTH positive control and PTH produced in CFPS prevented accurate detection of binding. PTH is slightly smaller than the molecular weight cutoff for this BLI instrument.

FIG. 11a-11b. Anti-microbial peptides can also be purified using the described method and are active in the prevention of growth of E. coli MG1655. (a) A Coomassie SDS-PAGE gel shows recovery of anti-microbial peptides (purple arrows) after the Strep-SUMO purification procedure described in FIG. 3a. (b) Melittin inhibits growth of E. coli MG1655 during overnight incubation in a 384 well plate. Error bars represent 1 s.d. of (n=2) replicates. Error bars not shown are smaller than the symbol.

FIG. 12a-12c. Exemplary His purification screen. The purification of the hormone panel liraglutide, GLP-1, growth hormone, and parathyroid hormone, was tested in a scheme where a 6×His affinity purification tag was used to purify the expressed constructs with Ni-NTA magnetic beads. Instead of standard imidazole elution, Ulp-1 was used to elute the tagless peptide hormone from the magnetic beads (FIG. 3a). In this example, Ulp-1 was also His tagged and should not be present after elution. The expected masses were confirmed by liquid chromatography in tandem with mass spectrometry on a Bruker Impact II Q-TOF. (a) Liraglutide, GLP-1, Growth Hormone, and Parathyroid Hormone were identified as the peptide hormones that eluted as tagless and intact with expected masses for the native structures being 3.5 kDa, 3.3 kDa, 22 kDa, and 9.4 kDa, respectively. (b) After C18 desalting, Liraglutide and GLP-1 were injected onto a peptide column and analyzed by ESI-MS. Peaks corresponding to both peptides were observed in the +5, +4, and +3 charge states. The inset shows a zoom on the isotope distribution of the +5 charge state. The calculated monoisotopic masses match the theoretical masses within a mass error of 3 ppm. (c) After C18 desalting, Growth Hormone and Parathyroid Hormone were injected onto a protein column and analyzed by ESI-MS. After deconvolution with the Bruker maximum entropy deconvolution tool, intact protein was detected within 308 ppm mass error for GH and 1.06 ppm for parathyroid hormone. While there were multiple contaminating peaks as proof of principle, it confirms the results seen by gel electrorphoresis (data not shown).

FIG. 13a-13g. Alternate purification schemes were explored because of a lack of reproducibility with the His tag. (a) The purification scheme after lyophilization of samples was tested in 759 lysate and a lysate from a strain of Clm24 engineered with deletion of IpxM, an enzyme that catalyzes the synthesis of Lipid A from Lipid IVA to reduce the endotoxin burden of the strain (Emiola et al 2015, Stark et al 2021). T7 RNAP was added to the low endotoxin Clm24 strains in a standard form containing glycerol in the stock solution and a dialyzed version of T7 that eliminated the glycerol since glycerol reduces the activity of lyophilized extracts (data not shown). No intact peptide hormones were recovered from this experiment. Multiple experiments after this failed to reproduce the data in FIG. 12. It was hypothesized that the binding of Ulp protease to Ni-NTA beads negatively impacted the enzyme kinetics since SUMO-hormone fusions were observed in gels eluted with imidazole (data not shown). Therefore, purification schemes with both FLAG (b) and Strep (e) tags were tested. In the FLAG purification scheme, no product was observed in the Ulp-eluted (c) gel. (d) Only SUMO-GH was observed in the SDS eluted gel. Therefore, it was concluded that the anti-FLAG magnetic beads were not binding the FLAG tags on the produced protein well enough to endure purification. (f) The Ulp-eluted gel from the strep purification scheme shows recovery of tagless Liraglutide, GLP-1, GH and PTH. Arrows represent bands of tagless hormones. (g) The biotin elution gel shows that increasing Ulp concentration decreases the amount of SUMO-GH and SUMO-PTH that remains bound to the beads. Arrows represent the SUMO-hormone.

FIG. 14a-14b. Optimization of the Ulp-1 elution step. The elution step was the main source of irreproducibility. Therefore, to make this step as robust as possible, multiple optimization experiments were performed. Two additional experiments were performed in addition to the Ulp titration experiment in FIG. 12. (a) Previously, there were contaminating bands in the Ulp elution gels (FIG. 12f), most likely due to excess strep-SUMO saturating the streptactin magnetic beads. Therefore, the volume of streptactin beads added was increased from 30 μl to 50 μl. The contaminating bands at ˜15 kDa fade significantly with the increased binding capacity. The addition of Ni-NTA beads after the Ulp elution step was also tested to eliminate the His-tagged Ulp from the eluent. The Ulp is present at a concentration that is too low to see by Coomassie. However, contaminating bands also fade, likely due to non-specific binding. Biotin eluted samples are included as a control. These lanes contain a large amount of Strep-SUMO as expected. Only GH has incomplete cleavage as demonstrated by the band at ˜35 kDa. Arrows represent intact hormones and expected masses for these hormones are given on the bottom of the gel. (b) For the His purification protocol, the tubes could not be disrupted significantly during the Ulp elution step; therefore, tubes were laid flat and flipped manually every ˜15 min during the Ulp cleavage step. However, this is time consuming and hard to replicate run to run. Therefore, performing the spinning incubation on a benchtop rotator that spun at 18 rpm was attempted. There is no significant difference in the flipped and spun samples. Therefore, Ulp cleavage can be performed by spinning the tubes on a benchtop rotator.

### DETAILED DESCRIPTION

Disclosed herein is a workflow for expressing human hormones as protein fusions in lyophilized and non-lyophilized cell-free extract and a purification and cleavage strategy that yields tagless hormones.

### Definitions

To aid in understanding the invention, several terms are defined below.

Unless defined otherwise, all technical and scientific terms used herein have the same meaning as commonly understood by one of skill in the art. Although any methods and materials similar to or equivalent to those described herein can be used in the practice or testing of the claims, the exemplary methods and materials are described herein.

Moreover, reference to an element by the indefinite article “a” or “an” does not exclude the possibility that more than one element is present, unless the context clearly requires that there be one and only one element. The indefinite article “a” or “an” thus usually means “at least one.”

The term “about” means within a statistically meaningful range of a value or values such as a stated concentration, length, molecular weight, pH, time frame, temperature, pressure or volume. Such a value or range can be within an order of magnitude, typically within 20%, more typically within 10%, and even more typically within 5% of a given value or range. The allowable variation encompassed by “about” will depend upon the particular system under study.

The terms “comprising,” “having,” “including,” and “containing” are to be construed as open-ended terms (i.e., meaning “including, but not limited to,”) unless otherwise noted.

Recitation of ranges of values herein are merely intended to serve as a shorthand method of referring individually to each separate value falling within the range, and includes the endpoint boundaries defining the range, unless otherwise indicated herein, and each separate value is incorporated into the specification as if it were individually recited herein.

The terms “nucleic acid” and “oligonucleotide,” as used herein, refer to polydeoxyribonucleotides (containing 2-deoxy-D-ribose), polyribonucleotides (containing D-ribose), and to any other type of polynucleotide that is an N glycoside of a purine or pyrimidine base. There is no intended distinction in length between the terms “nucleic acid”, “oligonucleotide” and “polynucleotide”, and these terms will be used interchangeably. These terms refer only to the primary structure of the molecule. Thus, these terms include double- and single-stranded DNA, as well as double- and single-stranded RNA. For use in the present invention, an oligonucleotide also can comprise nucleotide analogs in which the base, sugar or phosphate backbone is modified as well as non-purine or non-pyrimidine nucleotide analogs.

Oligonucleotides can be prepared by any suitable method, including direct chemical synthesis by a method such as the phosphotriester method of Narang et al., 1979, Meth. Enzymol. 68:90-99; the phosphodiester method of Brown et al., 1979, Meth. Enzymol. 68:109-151; the diethylphosphoramidite method of Beaucage et al., 1981, Tetrahedron Letters 22:1859-1862; and the solid support method of U.S. Pat. No. 4,458,066, each incorporated herein by reference. A review of synthesis methods of conjugates of oligonucleotides and modified nucleotides is provided in Goodchild, 1990, Bioconjugate Chemistry 1 (3): 165-187, incorporated herein by reference.

The term “primer,” as used herein, refers to an oligonucleotide capable of acting as a point of initiation of DNA synthesis under suitable conditions. Such conditions include those in which synthesis of a primer extension product complementary to a nucleic acid strand is induced in the presence of four different nucleoside triphosphates and an agent for extension (for example, a DNA polymerase or reverse transcriptase) in an appropriate buffer and at a suitable temperature.

A primer is preferably a single-stranded DNA. The appropriate length of a primer depends on the intended use of the primer but typically ranges from about 6 to about 225 nucleotides, including intermediate ranges, such as from 15 to 35 nucleotides, from 18 to 75 nucleotides and from 25 to 150 nucleotides. Short primer molecules generally require cooler temperatures to form sufficiently stable hybrid complexes with the template. A primer need not reflect the exact sequence of the template nucleic acid, but must be sufficiently complementary to hybridize with the template. The design of suitable primers for the amplification of a given target sequence is well known in the art and described in the literature cited herein.

Primers can incorporate additional features which allow for the detection or immobilization of the primer but do not alter the basic property of the primer, that of acting as a point of initiation of DNA synthesis. For example, primers may contain an additional nucleic acid sequence at the 5′ end which does not hybridize to the target nucleic acid, but which facilitates cloning or detection of the amplified product, or which enables transcription of RNA (for example, by inclusion of a promoter) or translation of protein (for example, by inclusion of a 5′-UTR, such as an Internal Ribosome Entry Site (IRES) or a 3′-UTR element, such as a poly (A) n sequence, where n is in the range from about 20 to about 200). The region of the primer that is sufficiently complementary to the template to hybridize is referred to herein as the hybridizing region.

The term “promoter” refers to a cis-acting DNA sequence that directs RNA polymerase and other trans-acting transcription factors to initiate RNA transcription from the DNA template that includes the cis-acting DNA sequence.

The terms “target”, “target sequence”, “target region”, and “target nucleic acid”, as used herein, are synonymous and refer to a region or sequence of a nucleic acid which is to be amplified, sequenced or detected.

The term “hybridization”, as used herein, refers to the formation of a duplex structure by two single-stranded nucleic acids due to complementary base pairing. Hybridization can occur between fully complementary nucleic acid strands or between “substantially complementary” nucleic acid strands that contain minor regions of mismatch. Conditions under which hybridization of fully complementary nucleic acid strands is strongly preferred are referred to as “stringent hybridization conditions” or “sequence-specific hybridization conditions”. Stable duplexes of substantially complementary sequences can be achieved under less stringent hybridization conditions; the degree of mismatch tolerated can be controlled by suitable adjustment of the hybridization conditions. Those skilled in the art of nucleic acid technology can determine duplex stability empirically considering a number of variables including, for example, the length and base pair composition of the oligonucleotides, ionic strength, and incidence of mismatched base pairs, following the guidance provided by the art (see, e.g., Sambrook et al., 1989, Molecular Cloning-A Laboratory Manual, Cold Spring Harbor Laboratory, Cold Spring Harbor, New York; Wetmur, 1991, Critical Review in Biochem. and Mol. Biol. 26 (3/4): 227-259; and Owczarzy et al., 2008, Biochemistry, 47:5336-5353, which are incorporated herein by reference).

The term “amplification reaction” refers to any chemical reaction, including an enzymatic reaction, which results in increased copies of a template nucleic acid sequence or results in transcription of a template nucleic acid. Amplification reactions include reverse transcription, the polymerase chain reaction (PCR), including Real Time PCR (see U.S. Pat. Nos. 4,683,195 and 4,683,202; PCR Protocols: A Guide to Methods and Applications (Innis et al., eds, 1990)), and the ligase chain reaction (LCR) (see Barany et al., U.S. Pat. No. 5,494,810). Exemplary “amplification reactions conditions” or “amplification conditions” typically comprise either two or three step cycles. Two-step cycles have a high temperature denaturation step followed by a hybridization/elongation (or ligation) step. Three step cycles comprise a denaturation step followed by a hybridization step followed by a separate elongation step.

As used herein, a “polymerase” refers to an enzyme that catalyzes the polymerization of nucleotides. “DNA polymerase” catalyzes the polymerization of deoxyribonucleotides. Known DNA polymerases include, for example, Pyrococcus furiosus (Pfu) DNA polymerase, E. coli DNA polymerase I, T7 DNA polymerase and Thermus aquaticus (Taq) DNA polymerase, among others. “RNA polymerase” catalyzes the polymerization of ribonucleotides. The foregoing examples of DNA polymerases are also known as DNA-dependent DNA polymerases. RNA-dependent DNA polymerases also fall within the scope of DNA polymerases. Reverse transcriptase, which includes viral polymerases encoded by retroviruses, is an example of an RNA-dependent DNA polymerase. Known examples of RNA polymerase (“RNAP”) include, for example, T3 RNA polymerase, T7 RNA polymerase, SP6 RNA polymerase and E. coli RNA polymerase, among others. The foregoing examples of RNA polymerases are also known as DNA-dependent RNA polymerase. The polymerase activity of any of the above enzymes can be determined by means well known in the art.

As used herein, a primer is “specific,” for a target sequence if, when used in an amplification reaction under sufficiently stringent conditions, the primer hybridizes primarily to the target nucleic acid. Typically, a primer is specific for a target sequence if the primer-target duplex stability is greater than the stability of a duplex formed between the primer and any other sequence found in the sample. One of skill in the art will recognize that various factors, such as salt conditions as well as base composition of the primer and the location of the mismatches, will affect the specificity of the primer, and that routine experimental confirmation of the primer specificity will be needed in many cases. Hybridization conditions can be chosen under which the primer can form stable duplexes only with a target sequence. Thus, the use of target-specific primers under suitably stringent amplification conditions enables the selective amplification of those target sequences that contain the target primer binding sites.

As used herein, “expression template” refers to a nucleic acid that serves as substrate for transcribing at least one RNA that can be translated into a polypeptide or protein. Expression templates include nucleic acids composed of DNA or RNA. Suitable sources of DNA for use a nucleic acid for an expression template include genomic DNA, cDNA and RNA that can be converted into cDNA. Genomic DNA, cDNA and RNA can be from any biological source, such as a tissue sample, a biopsy, a swab, sputum, a blood sample, a fecal sample, a urine sample, a scraping, among others. The genomic DNA, cDNA and RNA can be from host cell or virus origins and from any species, including extant and extinct organisms. As used herein, “expression template” and “transcription template” have the same meaning and are used interchangeably.

As used herein, “translation template” refers to an RNA product, typically produced by transcription from an expression template, that can be used by ribosomes to synthesize polypeptide or protein.

The term “reaction mixture” or “reaction composition” as used herein, refers to a solution containing reagents necessary to carry out a given reaction. A reaction mixture is referred to as complete if it contains all reagents necessary to enable the reaction, and incomplete if it contains only a subset of the necessary reagents. Components for a reaction mixture may be stored separately in separate container, each containing one or more of the total components. Components may be packaged separately for commercialization and useful commercial kits may contain one or more of the reaction components for a reaction mixture.

An “amplification reaction mixture”, which refers to a solution containing reagents necessary to carry out an amplification reaction, typically contains oligonucleotide primers and a DNA polymerase in a suitable buffer.

A “PCR reaction mixture”, which refers to a solution containing the reagents necessary to carry out a PCR reaction, typically contains DNA polymerase, dNTPs, and a divalent metal cation in a suitable buffer.

A “cell-free protein synthesis (CFPS) reaction mixture”, or a “CFPS reaction composition” which refers to a solution containing the reagents necessary to carry out CFPS, typically contains a crude or partially-purified bacterial or yeast extract, an RNA translation template, and a suitable reaction buffer for promoting cell-free protein synthesis from the RNA translation template. In some aspects, the CFPS reaction mixture can include exogenous RNA translation template. In other aspects, the CFPS reaction mixture can include a DNA expression template encoding an open reading frame operably linked to a promoter element for a DNA-dependent RNA polymerase. By way of example, an RNA translation template may be encoded on a vector. In these and other aspects, the CFPS reaction mixture can also include a DNA-dependent RNA polymerase to direct transcription of an RNA translation template encoding the open reading frame. In these and other aspects, additional NTP's and divalent cation cofactor can be included in the CFPS reaction mixture.

In some embodiments, the CFPS reaction mixture includes a polymerase. The polymerase may be endogenous to the lysate, or the polymerase may be exogenous, and may be added, either as a purified polymerase or as part of a different lysate. By way of example, but not by way of limitation, in some embodiments, a polymerase, such as T7 polymerase, is added to the CFPS reaction mixture (exogenous). In some embodiments, a CFPS reaction mixture is lyophilized, e.g., for long term storage.

By way of example but not by way of limitation, in some embodiments, a CFPS reaction mixture comprises a lysate, e.g., a bacterial lysate, one or more lyoprotectants, and a buffer comprising, for example, HEPES, phosphate, Bis-Tris 10, and magnesium. In some embodiments, the RNA translation template is also present in the CFPS reaction mixture, and can be endogenous to the lysate or can be an added component. In some embodiments, the CFPS reaction mixture is lyophilized.

A “secondary reaction mixture,” which refers to a solution containing the reagents necessary to carry out an enzyme-mediated biosynthetic steps, typically includes a feedstock that reacts in the presence of the enzyme to produce a final or intermediate product in the metabolic or biosynthetic pathway of interest. A secondary reaction mixture may optionally contain a cofactor, e.g. coenzyme-A, NAD, ATP, or a buffer.

The polynucleotide sequences contemplated herein may be present in expression vectors. For example, the vectors may comprise: (a) a polynucleotide encoding an ORF of a protein; (b) a polynucleotide that expresses an RNA that directs RNA-mediated binding, nicking, and/or cleaving of a target DNA sequence; and both (a) and (b). The polynucleotide present in the vector may be operably linked to a prokaryotic or eukaryotic promoter. “Operably linked” refers to the situation in which a first nucleic acid sequence is placed in a functional relationship with a second nucleic acid sequence. For instance, a promoter is operably linked to a coding sequence if the promoter affects the transcription or expression of the coding sequence. Operably linked DNA sequences may be in close proximity or contiguous and, where necessary to join two protein coding regions, in the same reading frame. Vectors contemplated herein may comprise a heterologous promoter (e.g., a eukaryotic or prokaryotic promoter) operably linked to a polynucleotide that encodes a protein. A “heterologous promoter” refers to a promoter that is not the native or endogenous promoter for the protein or RNA that is being expressed. Vectors as disclosed herein may include plasmid vectors.

As used herein, “expression” refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

In certain exemplary embodiments, vectors such as, for example, expression vectors, containing a nucleic acid encoding one or more rRNAs or reporter polypeptides and/or proteins described herein are provided. As used herein, the term “vector” refers to a nucleic acid molecule capable of transporting another nucleic acid to which it has been linked. One type of vector is a “plasmid,” which refers to a circular double stranded DNA loop into which additional DNA segments can be ligated. Such vectors are referred to herein as “expression vectors.” In general, expression vectors of utility in recombinant DNA techniques are often in the form of plasmids. In the present specification, “plasmid” and “vector” can be used interchangeably. However, the disclosed methods and compositions are intended to include such other forms of expression vectors, such as viral vectors (e.g., replication defective retroviruses, adenoviruses and adeno-associated viruses), which serve equivalent functions.

In certain exemplary embodiments, the recombinant expression vectors comprise a nucleic acid sequence (e.g., a nucleic acid sequence encoding one or more rRNAs or reporter polypeptides and/or proteins described herein) in a form suitable for expression of the nucleic acid sequence in one or more of the methods described herein, which means that the recombinant expression vectors include one or more regulatory sequences which is operatively linked to the nucleic acid sequence to be expressed. Within a recombinant expression vector, “operably linked” is intended to mean that the nucleotide sequence encoding one or more rRNAs or reporter polypeptides and/or proteins described herein is linked to the regulatory sequence(s) in a manner which allows for expression of the nucleotide sequence (e.g., in an in vitro transcription and/or translation system). The term “regulatory sequence” is intended to include promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990).

Oligonucleotides and polynucleotides may optionally include one or more non-standard nucleotide(s), nucleotide analog(s) and/or modified nucleotides. Examples of modified nucleotides include, but are not limited to diaminopurine, S2T, 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-D46-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, 2,6-diaminopurine and the like. Nucleic acid molecules may also be modified at the base moiety (e.g., at one or more atoms that typically are available to form a hydrogen bond with a complementary nucleotide and/or at one or more atoms that are not typically capable of forming a hydrogen bond with a complementary nucleotide), sugar moiety or phosphate backbone.

The terms “polynucleotide,” “polynucleotide sequence,” “nucleic acid” and “nucleic acid sequence” refer to a nucleotide, oligonucleotide, polynucleotide (which terms may be used interchangeably), or any fragment thereof. These phrases also refer to DNA or RNA of genomic, natural, or synthetic origin (which may be single-stranded or double-stranded and may represent the sense or the antisense strand).

Regarding polynucleotide sequences, the terms “percent identity” and “% identity” refer to the percentage of residue matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences. Percent identity for a nucleic acid sequence may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST), which is available from several sources, including the NCBI, Bethesda, Md., at its website. The BLAST software suite includes various sequence analysis programs including “blastn,” that is used to align a known polynucleotide sequence with other polynucleotide sequences from a variety of databases. Also available is a tool called “BLAST 2 Sequences” that is used for direct pairwise comparison of two nucleotide sequences. “BLAST 2 Sequences” can be accessed and used interactively at the NCBI website. The “BLAST 2 Sequences” tool can be used for both blastn and blastp (discussed above).

Regarding polynucleotide sequences, percent identity may be measured over the length of an entire defined polynucleotide sequence, for example, as defined by a particular SEQ ID number, or may be measured over a shorter length, for example, over the length of a fragment taken from a larger, defined sequence, for instance, a fragment of at least 20, at least 30, at least 40, at least 50, at least 70, at least 100, or at least 200 contiguous nucleotides. Such lengths are exemplary only, and it is understood that any fragment length supported by the sequences shown herein, in the tables, figures, or Sequence Listing, may be used to describe a length over which percentage identity may be measured.

Regarding polynucleotide sequences, “variant,” “mutant,” or “derivative” may be defined as a nucleic acid sequence having at least 50% sequence identity to the particular nucleic acid sequence over a certain length of one of the nucleic acid sequences using blastn with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences”, FEMS Microbiol Lett. 174:247-250). Such a pair of nucleic acids may show, for example, at least 60%, at least 70%, at least 80%, at least 85%, at least 90%, at least 91%, at least 92%, at least 93%, at least 94%, at least 95%, at least 96%, at least 97%, at least 98%, or at least 99% or greater sequence identity over a certain defined length.

Nucleic acid sequences that do not show a high degree of identity may nevertheless encode similar amino acid sequences due to the degeneracy of the genetic code where multiple codons may encode for a single amino acid. It is understood that changes in a nucleic acid sequence can be made using this degeneracy to produce multiple nucleic acid sequences that all encode substantially the same protein. For example, polynucleotide sequences as contemplated herein may encode a protein and may be codon-optimized for expression in a particular host. In the art, codon usage frequency tables have been prepared for a number of host organisms including humans, mouse, rat, pig, E. coli, plants, and other host cells.

A “recombinant nucleic acid” is a sequence that is not naturally occurring or has a sequence that is made by an artificial combination of two or more otherwise separated segments of sequence. This artificial combination is often accomplished by chemical synthesis or, more commonly, by the artificial manipulation of isolated segments of nucleic acids, e.g., by genetic engineering techniques known in the art. The term recombinant includes nucleic acids that have been altered solely by addition, substitution, or deletion of a portion of the nucleic acid. Frequently, a recombinant nucleic acid may include a nucleic acid sequence operably linked to a promoter sequence. Such a recombinant nucleic acid may be part of a vector that is used, for example, to transform a cell.

The nucleic acids disclosed herein may be “substantially isolated or purified.” The term “substantially isolated or purified” refers to a nucleic acid that is removed from its natural environment, and is at least 60% free, preferably at least 75% free, and more preferably at least 90% free, even more preferably at least 95% free from other components with which it is naturally associated.

### Peptides, Polypeptides, Proteins, and Synthesis Methods

As described herein, the present compositions, kits, and methods are useful to produce proteins. As used herein, the terms “peptide,” “polypeptide,” and “protein,” refer to molecules comprising a chain a polymer of amino acid residues joined by amide linkages. The term “amino acid residue,” includes but is not limited to amino acid residues contained in the group consisting of alanine (Ala or A), cysteine (Cys or C), aspartic acid (Asp or D), glutamic acid (Glu or E), phenylalanine (Phe or F), glycine (Gly or G), histidine (His or H), isoleucine (Ile or I), lysine (Lys or K), leucine (Leu or L), methionine (Met or M), asparagine (Asn or N), proline (Pro or P), glutamine (Gln or Q), arginine (Arg or R), serine (Ser or S), threonine (Thr or T), valine (Val or V), tryptophan (Trp or W), and tyrosine (Tyr or Y) residues. The term “amino acid residue” also may include nonstandard or unnatural amino acids. The term “amino acid residue” may include alpha-, beta-, gamma-, and delta-amino acids.

In some embodiments, the term “amino acid residue” may include nonstandard or unnatural amino acid residues contained in the group consisting of homocysteine, 2-Aminoadipic acid, N-Ethylasparagine, 3-Aminoadipic acid, Hydroxylysine, β-alanine, β-Amino-propionic acid, allo-Hydroxylysine acid, 2-Aminobutyric acid, 3-Hydroxyproline, 4-Aminobutyric acid, 4-Hydroxyproline, piperidinic acid, 6-Aminocaproic acid, Isodesmosine, 2-Aminoheptanoic acid, allo-Isoleucine, 2-Aminoisobutyric acid, N-Methylglycine, sarcosine, 3-Aminoisobutyric acid, N-Methylisoleucine, 2-Aminopimelic acid, 6-N-Methyllysine, 2,4-Diaminobutyric acid, N-Methylvaline, Desmosine, Norvaline, 2,2′-Diaminopimelic acid, Norleucine, 2,3-Diaminopropionic acid, Ornithine, and N-Ethylglycine. The term “amino acid residue” may include L isomers or D isomers of any of the aforementioned amino acids.

Other examples of nonstandard or unnatural amino acids include, but are not limited, to a p-acetyl-L-phenylalanine, a p-iodo-L-phenylalanine, an O-methyl-L-tyrosine, a p-propargyloxyphenylalanine, a p-propargyl-phenylalanine, an L-3-(2-naphthyl) alanine, a 3-methyl-phenylalanine, an O-4-allyl-L-tyrosine, a 4-propyl-L-tyrosine, a tri-O-acetyl-GlcNAcpβ-serine, an L-Dopa, a fluorinated phenylalanine, an isopropyl-L-phenylalanine, a p-azido-L-phenylalanine, a p-acyl-L-phenylalanine, a p-benzoyl-L-phenylalanine, an L-phosphoserine, a phosphonoserine, a phosphonotyrosine, a p-bromophenylalanine, a p-amino-L-phenylalanine, an isopropyl-L-phenylalanine, an unnatural analogue of a tyrosine amino acid; an unnatural analogue of a glutamine amino acid; an unnatural analogue of a phenylalanine amino acid; an unnatural analogue of a serine amino acid; an unnatural analogue of a threonine amino acid; an unnatural analogue of a methionine amino acid; an unnatural analogue of a leucine amino acid; an unnatural analogue of a isoleucine amino acid; an alkyl, aryl, acyl, azido, cyano, halo, hydrazine, hydrazide, hydroxyl, alkenyl, alkynl, ether, thiol, sulfonyl, seleno, ester, thioacid, borate, boronate, 19ufa19hor, phosphono, phosphine, heterocyclic, enone, imine, aldehyde, hydroxylamine, keto, or amino substituted amino acid, or a combination thereof; an amino acid with a photoactivatable cross-linker; a spin-labeled amino acid; a fluorescent amino acid; a metal binding amino acid; a metal-containing amino acid; a radioactive amino acid; a photocaged and/or photoisomerizable amino acid; a biotin or biotin-analogue containing amino acid; a keto containing amino acid; an amino acid comprising polyethylene glycol or polyether; a heavy atom substituted amino acid; a chemically cleavable or photocleavable amino acid; an amino acid with an elongated side chain; an amino acid containing a toxic group; a sugar substituted amino acid; a carbon-linked sugar-containing amino acid; a redox-active amino acid; an a-hydroxy containing acid; an amino thio acid; an α,α disubstituted amino acid; a β-amino acid; a γ-amino acid, a cyclic amino acid other than proline or histidine, and an aromatic amino acid other than phenylalanine, tyrosine or tryptophan.

It is understood that natural and/or non-natural amino acids may be added to a CFPS reaction mixture for which components (e.g., nucleic acid templates, ribosomes, etc.) are provided to produce proteins comprising any of the natural or non-natural amino acids.

As used herein, the terms “peptide”, “protein”, and “polypeptide” are used interchangeably, and refer to a polymer of amino acids. In some embodiments, a polypeptide as contemplated herein may include no more than about 2, 3, 4, 5, 6, 7, 8, 9, 10, 11, 12, 13, 14, 15, 16, 17, 18, 19, or 20 amino acids. A polypeptide, also referred to as a protein, may comprise >100 amino acids. A polypeptide, as contemplated herein, may comprise, but is not limited to, 100, 101, 102, 103, 104, 105, about 110, about 120, about 130, about 140, about 150, about 160, about 170, about 180, about 190, about 200, about 210, about 220, about 230, about 240, about 250, about 275, about 300, about 325, about 350, about 375, about 400, about 425, about 450, about 475, about 500, about 525, about 550, about 575, about 600, about 625, about 650, about 675, about 700, about 725, about 750, about 775, about 800, about 825, about 850, about 875, about 900, about 925, about 950, about 975, about 1000, about 1100, about 1200, about 1300, about 1400, about 1500, about 1750, about 2000, about 2250, about 2500 or more amino acid residues.

A peptide as contemplated herein may be further modified to include non-amino acid moieties. Modifications may include but are not limited to acylation (e.g., O-acylation (esters), N-acylation (amides), S-acylation (thioesters)), acetylation (e.g., the addition of an acetyl group, either at the N-terminus of the protein or at lysine residues), formylation lipoylation (e.g., attachment of a lipoate, a C8 functional group), myristoylation (e.g., attachment of myristate, a C14 saturated acid), palmitoylation (e.g., attachment of palmitate, a C16 saturated acid), alkylation (e.g., the addition of an alkyl group, such as an methyl at a lysine or arginine residue), isoprenylation or prenylation (e.g., the addition of an isoprenoid group such as farnesol or geranylgeraniol), amidation at C-terminus, glycosylation (e.g., the addition of a glycosyl group to either asparagine, hydroxylysine, serine, or threonine, resulting in a glycoprotein). Distinct from glycation, which is regarded as a nonenzymatic attachment of sugars, polysialylation (e.g., the addition of polysialic acid), glypiation (e.g., glycosylphosphatidylinositol (GPI) anchor formation, hydroxylation, iodination (e.g., of thyroid hormones), and phosphorylation (e.g., the addition of a phosphate group, usually to serine, tyrosine, threonine or histidine).

A modified amino acid sequence that is disclosed herein may include a deletion in one or more amino acids. As utilized herein, a “deletion” means the removal of one or more amino acids relative to the native amino acid sequence. The modified amino acid sequences that are disclosed herein may include an insertion of one or more amino acids. As utilized herein, an “insertion” means the addition of one or more amino acids to a native amino acid sequence. The modified amino acid sequences that are disclosed herein may include a substitution of one or more amino acids. As utilized herein, a “substitution” means replacement of an amino acid of a native amino acid sequence with an amino acid that is not native to the amino acid sequence.

A “variant,” “mutant,” or “derivative” of a reference polypeptide sequence may include a deletion relative to the reference polypeptide sequence.

Regarding polypeptide or protein “fragments,” a “fragment” is a portion of an amino acid sequence which is identical in sequence to but shorter in length than a reference sequence. A fragment may comprise up to the entire length of the reference sequence, minus at least one amino acid residue. For example, a fragment may comprise from 5 to 1000 contiguous amino acid residues of a reference polypeptide, respectively. In some embodiments, a fragment may comprise at least 5, 10, 15, 20, 25, 30, 40, 50, 60, 70, 80, 90, 100, 150, 250, or 500 contiguous amino acid residues of a reference polypeptide. Fragments may be preferentially selected from certain regions of a molecule. The term “at least a fragment” encompasses the full-length polypeptide. A fragment may include an N-terminal truncation, a C-terminal truncation, or both truncations relative to the full-length protein. A “variant,” “mutant,” or “derivative” of a reference polypeptide sequence may include a fragment of the reference polypeptide sequence.

Regarding proteins, the words “insertion” and “addition” refer to changes in an amino acid sequence resulting in the addition of one or more amino acid residues. An insertion or addition may refer to 1, 2, 3, 4, 5, 10, 20, 30, 40, 50, 60, 70, 80, 90, 100, 150, 200, or more amino acid residues. A “variant,” “mutant,” or “derivative” of a reference polypeptide sequence may include an insertion or addition relative to the reference polypeptide sequence. A variant of a protein may have N-terminal insertions, C-terminal insertions, internal insertions, or any combination of N-terminal insertions, C-terminal insertions, and internal insertions.

Regarding proteins, the phrases “percent identity” and “% identity,” refer to the percentage of residue matches between at least two amino acid sequences aligned using a standardized algorithm. Methods of amino acid sequence alignment are well-known. Some alignment methods take into account conservative amino acid substitutions. Such conservative substitutions, explained in more detail below, generally preserve the charge and hydrophobicity at the site of substitution, thus preserving the structure (and therefore function) of the polypeptide. Percent identity for amino acid sequences may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST), which is available from several sources, including the NCBI, Bethesda, Md., at its website. The BLAST software suite includes various sequence analysis programs including “blastp,” that is used to align a known amino acid sequence with other amino acids sequences from a variety of databases.

Regarding proteins, percent identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

Regarding proteins, the amino acid sequences of variants, mutants, or derivatives as contemplated herein may include conservative amino acid substitutions relative to a reference amino acid sequence. For example, a variant, mutant, or derivative protein may include conservative amino acid substitutions relative to a reference molecule. “Conservative amino acid substitutions” are those substitutions that are a substitution of an amino acid for a different amino acid where the substitution is predicted to interfere least with the properties of the reference polypeptide. In other words, conservative amino acid substitutions substantially conserve the structure and the function of the reference polypeptide. The following table provides a list of exemplary conservative amino acid substitutions which are contemplated herein:

| Original | |
|---|---|
| Residue | Conservative Substitution |
| Ala | Gly, Ser |
| Arg | His, Lys |
| Asn | Asp, Gln, His |
| Asp | Asn, Glu |
| Cys | Ala, Ser |
| Gln | Asn, Glu, His |
| Glu | Asp, Gln, His |
| Gly | Ala |
| His | Asn, Arg, Gln, Glu |
| Ile | Leu, Val |
| Leu | Ile, Val |
| Lys | Arg, Gln, Glu |
| Met | Leu, Ile |
| Phe | His, Met, Leu, Trp, Tyr |
| Ser | Cys, Thr |
| Thr | Ser, Val |
| Trp | Phe, Tyr |
| Tyr | His, Phe, Trp |
| Val | Ile, Leu, Thr |

Conservative amino acid substitutions generally maintain (a) the structure of the polypeptide backbone in the area of the substitution, for example, as a beta sheet or alpha helical conformation, (b) the charge or hydrophobicity of the molecule at the site of the substitution, and/or (c) the bulk of the side chain. Non-conservative amino acids typically disrupt (a) the structure of the polypeptide backbone in the area of the substitution, for example, as a beta sheet or alpha helical conformation, (b) the charge or hydrophobicity of the molecule at the site of the substitution, and/or (c) the bulk of the side chain.

Disclosed proteins, mutants, or variants, described herein may have one or more functional or biological activities exhibited by a reference polypeptide (e.g., one or more functional or biological activities exhibited by wild-type protein). In some embodiments, the activity of the variant or mutant protein may have an activity that is enhanced, as compared to a comparable wild-type or control enzyme, or may have an alternative or a modified activity as compared to a comparable or wild-type or control enzyme.

In some embodiments, a protein comprises a fusion protein. As used herein, fusion protein refers to a protein created through the joining of two or more genes, or pieces of genes (e.g., gene fragments), that originally coded for separate proteins. In some embodiments, one part of a fusion protein may comprise a tag, or protein sequence that is useful, for example, to identify and/or isolate/purify the protein. By way of example, but not by way of limitation, exemplary protein tags (also termed in the art as peptide tags, purification tags, and affinity tags), include His tags, FLAG tags, E-tags, HA-tags, Myc-tags, T7 tags, polyglutamate or polyarginine tags, glutathione-S-Transferase tags, twin-strep tags, superFLAG tags, Fc Tags, streptavidin, maltose binding protein (MBP), cellulose binding domain (CBD), green fluorescent protein (GFP), HaloTag, SpyTag, SnapTag, horseradish peroxidase (HRP), polyarginine, polyaspartate, and polycysteine tags, and the like. Protein tags are well known in the art, and the skilled artisan would be able to select a tag suitable for identification and/or isolation as needed. See e.g., Kimple et al., “Overview of Affinity Tags for Protein Purification,” Cur Protoc Protein Sci. 2013; 73: unit-9.9, incorporated herein by reference in its entirety.

In some embodiments, different parts of a fusion protein may be joined by a linker sequence, also termed herein a flexible linker. In some embodiments, a linker joins differ segments of a fusion protein to promote function of the fusion, e.g., reduce or eliminate possible steric hindrance, allow protein folding, enhance protein stability or conformation, and afford additional flexibility to the fusion protein. Linker sequences and their use are well known in the art, and the skilled artisan would be able to select one or more linker sequences suitable for fusion proteins of the present technology (e.g., to provide flexibility between a protein of interest and a cleavable linker in a fusion protein; to allow protein tag function for binding to its binding partner, etc.). In some embodiments, the linker may comprise a cleavable linker. In some embodiments, the linker may be cleaved enzymatically, or chemically.

In some embodiments, constructs disclosed herein include one or more stability or solubility enhancing moieties. In some embodiments, constructs disclosed herein include one or more internal fusion proteins (IFP). In some embodiments, a stability or solubility enhancing moiety or IFP is cleavable e.g., by an enzyme or chemical. In some embodiments, a stability or solubility enhancing moiety comprises a internal fusion protein (IFP). In some embodiments, the internal fusion protein comprises a small ubiquitin-related modifier (SUMO). In embodiments, the IFP, e.g., SUMO includes a cleavage site. By way of example, but not by way of limitation, additional IFPs that would serve a similar function (e.g., provide an internal cleavage site, and optionally, act as a stability/solubility enhancer), include maltose binding protein, the TEV protease recognition site peptide, chymosin propeptide, glutathione-S-transferase, ubiquitin, cleavage sites for enterokinase, caspases, factor Xa, and thrombin. Regarding stability and solubility enhancing moieties, such moieties may be useful for smaller peptides, e.g., peptide hormones, but may not be needed for more stable therapeutic proteins, such as growth hormone and leptin.

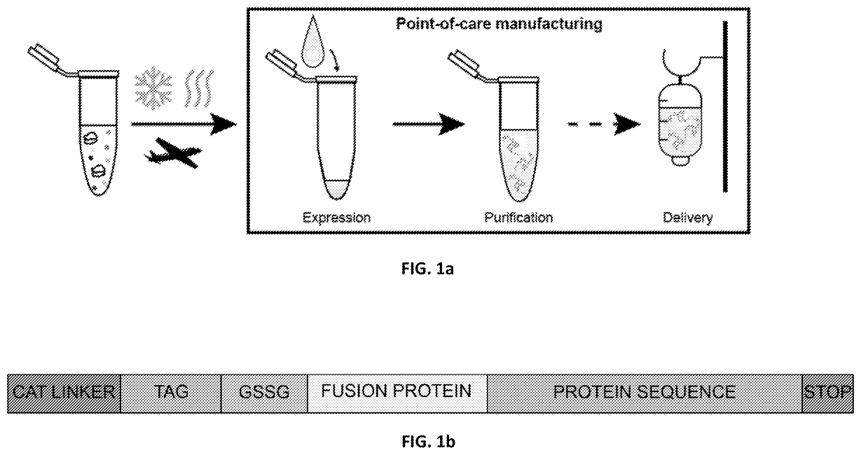

By way of example, but not by way of limitation, a fusion protein may comprise one or more of the following components: CAT enhancer; purification tag; a linker; a stability or solubility enhancing moiety; an internal fusion protein (e.g., a SUMO); a protein of interest, such as a hormone protein. In some embodiments the fusion protein comprises one or more of the proteins listed in Table 1 below. In some embodiments, the components are in order, from amino-terminus to carboxy terminus, as shown in FIG. 1b.

### Cell-Free Protein Synthesis (CFPS)

The components, systems, and methods disclosed herein may be applied to, or adapted to cell-free protein synthesis methods as known in the art. See, for example, U.S. Pat. Nos. 5,478,730; 5,556,769; 5,665,563; 6,168,931; 6,548,276; 6,869,774; 6,994,986; 7,118,883; 7,186,525; 7,189,528; 7,235,382; 7,338,789; 7,387,884; 7,399,610; 7,776,535; 7,817,794; 8,703,471; 8,298,759; 8,715,958; 8,734,856; 8,999,668; and 9,005,920. See also U.S. Published Application Nos. 2018/0016614, 2018/0016612, 2016/0060301, 2015-0259757, 2014/0349353, 2014-0295492, 2014-0255987, 2014-0045267, 2012-0171720, 2008-0138857, 2007-0154983, 2005-0054044, and 2004-0209321. See also U.S Published Application Nos. 2005-0170452; 2006-0211085; 2006-0234345; 2006-0252672; 2006-0257399; 2006-0286637; 2007-0026485; 2007-0178551. See also Published PCT International Application Nos. 2003/056914; 2004/013151; 2004/035605; 2006/102652; 2006/119987; and 2007/120932. See also Jewett, M. C., Hong, S. H., Kwon, Y. C., Martin, R. W., and Des Soye, B. J. 2014, “Methods for improved in vitro protein synthesis with proteins containing non-standard amino acids,” U.S. Patent Application Ser. No. 62/044,221; Jewett, M. C., Hodgman, C. E., and Gan, R. 2013, “Methods for yeast cell-free protein synthesis,” U.S. Patent Application Ser. No. 61/792,290; Jewett, M. C., J. A. Schoborg, and C. E. Hodgman. 2014, “Substrate Replenishment and Byproduct Removal Improve Yeast Cell-Free Protein Synthesis,” U.S. Patent Application Ser. No. 61/953,275; and Jewett, M. C., Anderson, M. J., Stark, J. C., Hodgman, C. E. 2015, “Methods for activating natural energy metabolism for improved yeast cell-free protein synthesis,” U.S. Patent Application Ser. No. 62/098,578. See also Guarino, C., & DeLisa, M. P. (2012). A prokaryote-based cell-free translation system that efficiently synthesizes glycoproteins. Glycobiology, 22 (5), 596-601. The contents of all of these references are incorporated in the present application by reference in their entireties.

As described above, in some embodiments, a “CFPS reaction mixture” typically may contain a crude or partially-purified cell extract (e.g., a yeast or bacterial extract), an RNA translation template, and a suitable reaction buffer for promoting cell-free protein synthesis from the RNA translation template. In some aspects, the CFPS reaction mixture can include exogenous RNA translation template. In other aspects, the CFPS reaction mixture can include a DNA expression template encoding an open reading frame operably linked to a promoter element for a DNA-dependent RNA polymerase. In these other aspects, the CFPS reaction mixture can also include a DNA-dependent RNA polymerase to direct transcription of an RNA translation template encoding the open reading frame. In these other aspects, additional NTP's and divalent cation cofactor can be included in the CFPS reaction mixture. In some embodiments, the CFPS reaction mixture is lyophilized. A reaction mixture is referred to as complete if it contains all reagents necessary to enable the reaction, and incomplete if it contains only a subset of the necessary reagents. It will be understood by one of ordinary skill in the art that reaction components are routinely stored as separate solutions, each containing a subset of the total components, for reasons of convenience, storage stability, or to allow for application-dependent adjustment of the component concentrations, and that reaction components are combined prior to the reaction to create a complete reaction mixture. Furthermore, it will be understood by one of ordinary skill in the art that reaction components are packaged separately for commercialization and that useful commercial kits may contain any subset of the reaction components of the invention.

The disclosed cell-free protein synthesis systems may utilize components that are crude and/or that are at least partially isolated and/or purified. As used herein, the term “crude” may mean components obtained by disrupting and lysing cells and, at best, minimally purifying the crude components from the disrupted and lysed cells, for example by centrifuging the disrupted and lysed cells and collecting the crude components from the supernatant and/or pellet after centrifugation. The term “isolated or purified” refers to components that are removed from their natural environment, and are at least 60% free, preferably at least 75% free, and more preferably at least 90% free, even more preferably at least 95% free from other components with which they are naturally associated.

In some embodiments, CFPS reactions include a crude or partially-purified cell extract. In some embodiments, the cells used to derive the crude or partially purified extract may be selected based on the presence or absence of specific endogenous biochemical pathways, and/or engineered biochemical pathways. For example, cells that direct carbon flux, prevent or minimize side product formation, and prevent or minimize promiscuous background activity may be advantageous as compared to other cells. In some embodiments, the cell is a prokaryotic cell (e.g., bacterial cell) or a eukaryotic cell (e.g., a yeast cell). In some embodiments, the cell is a prokaryotic cell and comprises and E. coli cell. In some embodiments, the E. coli cell comprises a modified E. coli cell, such as BL21, JST07, MB263, MP263sucD, and JC01. In some embodiments, the E. coli cell comprises JST07. In some embodiments, E. coli cells comprising BL21DE3 cells are the source of polymerase, such asT7 polymerase. In some embodiments, the cell is a bacterial cell and is modified for low endotoxin expression. By way of example, in some embodiments, the bacterial strain comprise an E. coli strain that includes the Clm24ΔIpxM modification (PglB-LpxE is overexpressed, the ETEC 078 biosynthesis pathway overexpressed; remodeled lipid A used to produce glycoconjugate proteins, such as vaccines). This bacterial strain is not grown with glucose.

For example, a CFPS reaction mixture may include a bacterial lysate, an expression template, a translation template, or both an expression template and a translation template. The expression template serves as a substrate for transcribing at least one RNA that can be translated into a sequence defined biopolymer (e.g., a polypeptide or protein). The translation template is an RNA product that can be used by ribosomes to synthesize the sequence defined biopolymer. In certain embodiments the platform comprises both the expression template and the translation template. In certain specific embodiments, the reaction mixture may comprise a coupled transcription/translation (“Tx/Tl”) system where synthesis of translation template and a sequence defined biopolymer from the same cellular extract.

The CFPS reaction mixture may comprise one or more polymerases capable of generating a translation template from an expression template. The polymerase may be supplied exogenously or may be supplied from the organism used to prepare the extract. In certain specific embodiments, the polymerase is expressed from a plasmid present in the organism used to prepare the extract and/or an integration site in the genome of the organism used to prepare the extract. In some embodiments, the polymerase is added to the CFPS reaction mixture. While T7 polymerase is exemplified herein, any polymerase may be used, so long as compatible promoter sequences have been provided. Such engineering would be routine to the skilled artisan.

Altering the physicochemical environment of the CFPS reaction to better mimic the cytoplasm can improve protein synthesis activity. The following parameters can be considered alone or in combination with one or more other components to improve robust CFPS reaction platforms based upon crude cellular extracts.

The temperature may be any temperature suitable for CFPS. Temperature may be in the general range from about 10° C. to about 40° C., including intermediate specific ranges within this general range, include from about 15° C. to about 35° C., from about 15° C. to about 30° C., from about 15° C. to about 25° C. In certain aspects, the reaction temperature can be about 15° C., about 16° C., about 17° C., about 18° C., about 19° C., about 20° C., about 21° C., about 22° C., about 23° C., about 24° C., about 25° C.

The reaction mixture may include any organic anion suitable for CFPS. In certain aspects, the organic anions can be glutamate, acetate, among others. In certain aspects, the concentration for the organic anions is independently in the general range from about 0 mM to about 200 mM, including intermediate specific values within this general range, such as about 0 mM, about 10 mM, about 20 mM, about 30 mM, about 40 mM, about 50 mM, about 60 mM, about 70 mM, about 80 mM, about 90 mM, about 100 mM, about 110 mM, about 120 mM, about 130 mM, about 140 mM, about 150 mM, about 160 mM, about 170 mM, about 180 mM, about 190 mM and about 200 mM, among others.

The reaction mixture may include any halide anion suitable for CFPS. In certain aspects the halide anion can be chloride, bromide, iodide, among others. A preferred halide anion is chloride. Generally, the concentration of halide anions, if present in the reaction, is within the general range from about 0 mM to about 200 mM, including intermediate specific values within this general range, such as those disclosed for organic anions generally herein.

The reaction mixture may include any organic cation suitable for CFPS. In certain aspects, the organic cation can be a polyamine, such as spermidine or putrescine, among others. Preferably polyamines are present in the CFPS reaction. In certain aspects, the concentration of organic cations in the reaction can be in the general about 0 mM to about 3 mM, about 0.5 mM to about 2.5 mM, about 1 mM to about 2 mM. In certain aspects, more than one organic cation can be present.

The reaction mixture may include any inorganic cation suitable for CFPS. For example, suitable inorganic cations can include monovalent cations, such as sodium, potassium, lithium, among others; and divalent cations, such as magnesium, calcium, manganese, among others. In certain aspects, the inorganic cation is magnesium. In such aspects, the magnesium concentration can be within the general range from about 1 mM to about 50 mM, including intermediate specific values within this general range, such as about 1 mM, about 2 mM, about 3 mM, about 5 mM, about 6 mM, about 7 mM, about 8 mM, about 9 mM, about 10 mM, among others. In some aspects, the concentration of inorganic cations can be within the specific range from about 4 mM to about 9 mM and more preferably, within the range from about 5 mM to about 7 mM. In some aspects, the concentration of inorganic cations by be about 8-10 mM.

The reaction mixture may include endogenous NTPs (i.e., NTPs that are present in the cell extract) and or exogenous NTPs (i.e., NTPs that are added to the reaction mixture). In certain aspects, the reaction uses ATP, GTP, CTP, and UTP. In certain aspects, the concentration of individual NTPs is within the range from about 0.1 mM to about 2 mM. In some embodiments, NTP are replaced with or supplemented with NMPs.

Buffers may be added, for example, to modulate or maintain the pH. Exemplary buffers include one or more of HEPES, Tris-Bis. The pH of the buffers can be adjusted, as is well known in the art. By way of example, in some embodiments, a Bis-Tris buffer of pH 5-10 may be used; in some embodiments, a HEPES buffer of pH 5-10 may be used. In some embodiments, the pH of a Bis-Tris buffer may be between 7-10 (e.g., 7.2). In some embodiments, the pH of a HEPES buffer may be between 7-10 (e.g., 7.2).

In some embodiments, phosphate is provided, for example, between about 0 and 100 mM. In some embodiments, phosphate is provided at between about 25-100 mM, or at about 75 mM.

The reaction mixture may include any alcohol suitable for CFPS. In certain aspects, the alcohol may be a polyol, and more specifically glycerol. In certain aspects the alcohol is between the general range from about 0% (v/v) to about 25% (v/v), including specific intermediate values of about 5% (v/v), about 10% (v/v) and about 15% (v/v), and about 20% (v/v), among others.

In certain exemplary embodiments, one or more of the methods described herein are performed in a vessel, e.g., a single, vessel. The term “vessel,” as used herein, refers to any container suitable for holding on or more of the reactants (e.g., for use in one or more transcription, translation, and/or secondary reaction steps) described herein. Examples of vessels include, but are not limited to, a microtitre plate, a test tube, a microfuge tube, a beaker, a flask, a multi-well plate, a cuvette, a flow system, a microfiber, a microscope slide and the like.

In some embodiments, the protein is isolated, purified, or concentrated from the CFPS reaction composition. In some embodiments, the protein is glycosylated.

### Applications and Advantages

The systems, methods, compositions, and components disclosed herein find use in numerous applications and advantages. Non-limiting examples include but are not limited to the following:

Point-of-care delivery of therapeutically relevant human hormones.

On-demand synthesis of hormone therapeutics.

On-site purification of therapeutics yielding tagless hormones.

On-site production of hormones circumvents need for cold chain storage.

On-demand expression and purification expand access of therapeutically relevant hormones to resource-limited settings.

Does not require living cells during the workflow.

Plug-and-play method where whichever therapeutic protein is needed can be chosen by addition of the corresponding plasmid.

Purification and cleavage strategy yields tagless hormones with identical amino acid sequence to endogenous form.

Current hormone production practices rely on centralized manufacturing and cold-chain distribution. The disclosed methods and compositions use lyophilized cell free extract, which is stable at room temperature.

The system is stable at room temperature for >2 weeks, and can be transported to the point-of-care and used for on-demand synthesis of the relevant therapeutic protein as well as purification and cleavage that yields hormones identical in amino acid sequence to what the human body naturally produces.

The invention offers a strategy for protein therapeutic manufacturing that is accessible to resource-limited settings. Expression of hormone-protein fusions from lyophilized cell-free extract enables point-of-care production and on-demand synthesis of the chosen hormone based on the plasmid with which the extract is rehydrated. It also circumvents the need for cold-chain distribution and provides a purification scheme that leverages fusion protein cleavage to allow for isolation of endogenous hormone on-site.

The present methods and compositions address many of the problems related to the prior art methods of hormone synthesis (e.g., chemo-enzymatic synthesis, continuous transcription and translation apparatuses, recombinant expression).

### Exemplary Embodiments

Embodiment 1. A method for cell-free expression of a hormone fusion protein (the protein), comprising: DNA design of genetic constructs encoding a therapeutic hormone, wherein the DNA construct comprises the structure of FIG. 1, Appendix I.

Embodiment 2. The method of embodiment 1, wherein the hormone fusion protein is expressed in a cell-free protein synthesis (CFPS) reaction.

Embodiment 3. The method of any of the previous embodiments, wherein the CFPS comprises genetically modified E. coli extract.

Embodiment 4. The method of any of the previous embodiments, wherein the extract facilitates disulfide bond formation.

Embodiment 5. The method of any of the previous embodiments, wherein the CFPS is lyophilized prior to use, and the method comprises rehydration of the CFPS.

Embodiment 6. The method of any of the previous embodiments, wherein the protein is expressed by the CFPS, and wherein the protein is evaluated by one or more methods comprising: fluorotect gel analysis, radioactivity assay, mass spectrometry.

Embodiment 7. The method of any of the previous embodiments, wherein the hormone fusion protein comprises a cleavable moiety, and the method comprises cleaving the cleavable moiety.

Embodiment 8. The method of any of the previous embodiments, wherein the hormone fusion protein comprises a purification tag, and the method comprises purifying the hormone fusion protein.

Embodiment 9. The method of any of the previous embodiments, wherein the cleavage moiety is positioned between the hormone, and the purification tag.

Embodiment 10. Purification and cleavage that yields tagless human hormones comprising one or more of the following steps: DNA design of genetic constructs with cleavable fusion protein intervening between purification tag and hormone; affinity-tag purification of cell-free expressed hormone-protein fusions; cleavage of protein and tag with free protease followed by addition of beads to bind up protease and allow capture of flow-through containing cleaved, tagless hormone; cleavage of protein and tag with bead-bound protease and capture of flow-through containing cleaved, tagless hormone; evaluation of tagless hormone by antibody recognition in ELISA; evaluation of tagless hormone by receptor binding with BLI;

Embodiment 11. The method any of the previous embodiments, where the hormone fusion protein comprises an internal fusion protein (IFP), wherein the IFP imparts one or more of stability, and solubility; and/or wherein the internal fusion protein is cleavable.

Embodiment 12. The method of any of the previous embodiments, wherein the internal fusion protein is TEV.

Embodiment 13. The method of any of the previous embodiments, wherein cell-free extracts are derived from other prokaryotes.

Embodiment 14. The method of any of the previous embodiments, wherein the cell free extract are derived from eukaryotic organisms.

Embodiment 15. The method of any of the previous embodiments, wherein multiple therapeutic proteins are expressed.

Embodiment 16. The method of any of the previous embodiments, wherein the therapeutic protein comprises a receptor binder.

Embodiment 17. The method embodiment 16, comprising a receptor binding assay, wherein the receptor binding is assessed by SPR, biolayer interferometry (BLI), and/or AlphaLISA to SPR to test receptor binding in vitro.

Embodiment 18. The method of any of the previous embodiments, wherein protein is isolated by immobilization.

Embodiment 19. The method of embodiment 15, comprising purifying the additional proteins.

### EXAMPLES

### Example 1: Point of Care Peptide Hormone Production Via Cell-Free Protein Synthesis

### Abstract

In resource-limited settings, it can be difficult to safely deliver sensitive biologic medicines to patients due to temperature and infrastructure constraints. Point-of-care drug manufacturing could circumvent these challenges because medicines would be produced locally and used on-demand. Peptide hormones are an important class of medications that can be used to treat a wide variety of diseases including diabetes, osteoporosis, and growth disorders. In this work, a platform was developed that uses cell-free protein synthesis (CFPS) and a 2-in-1 affinity purification and enzymatic cleavage scheme to synthesize a panel of peptide hormones. With this approach, temperature stable lyophilized CFPS reaction components can be shipped to resource-limited settings at ambient temperatures and rehydrated with the plasmid encoding a SUMOylated peptide hormone of interest when needed. Strep-Tactin® affinity purification and on-bead SUMO protease cleavage yields endogenous peptide hormones that are recognized by ELISA antibodies and that can bind their respective receptors. With further development to ensure proper biologic activity and patient safety, this platform could be used to manufacture valuable peptide hormone drugs at the point of care in resource-limited settings.

### Introduction

Peptide hormones are a class of biologic drugs with a range of therapeutic benefits, particularly in the treatment of chronic diseases, which disproportionately affect people living in resource-limited settings (1). Diseases like diabetes (2), obesity (3, 4), and osteoporosis (5) are commonly treated with peptide hormone biologics. GLP-1 receptor agonists, like semaglutide and liraglutide, have been used for the treatment of diabetes (6) and more recently, have been highlighted for their use in obesity (7, 8). Growth hormone is used for both congenital growth hormone deficiencies in children and deficiencies secondary to systemic diseases like chronic kidney disease and cancer (9-11). Parathyroid hormone can be used in the treatment of both osteoporosis (12) and hypoparathyroidism (13). Because these biologics are manufactured in centralized facilities, it makes delivery to patients in resource-limited settings difficult.

Current centralized manufacturing processes present challenges, highlighted by the COVID-19 pandemic, in both the transportation and storage of sensitive biologic materials. Packaging to minimize mechanical stresses and maintenance of cold chain temperatures, which requires both reliable energy sources and temperature monitors, are important considerations for the appropriate handling of sensitive biologic therapeutics (14). The high cost and need for reliable infrastructure of these measures makes it difficult to deliver these drugs safely and effectively to resource-limited settings. By nature, it is hard to accurately predict a priori the quantity of a drug that will be needed. Therefore, there tends to either be a shortage or an excess of each desired drug with current centralized manufacturing processes. When there is excess drug product that cannot be used before its expiration, it must be disposed of properly or risk contaminating water supplies (15), which adds an additional cost burden.

Cell-free protein synthesis (CFPS) can be used to address some of these challenges. CFPS is a technology that harvests the transcription and translation machinery from cells to produce peptides and proteins in vitro (16). CFPS is advantageous because it can be lyophilized for transportation and storage at ambient conditions (17) thus eliminating the need for maintenance of cold chain storage. After transportation and storage, CFPS reactions can be rehydrated on-demand at the point-of-care to synthesize the drug of interest (18, 19). Previous work has explored the idea of magistral drug production to produce a prostate specific membrane antigen, Y90-ibritumomab tiuxetan, and others in Bogota Columbia (20), a Francisella tularensis glycoconjugate vaccine (18), antimicrobial peptides, a diphtheria vaccine, nanobodies, DARPins (19), and granulocyte colony stimulating factor in a portable suitcase setup (21). There have also been numerous reviews and commentaries emphasizing the need to reimagine drug manufacturing away from current reliance on centralized facilities (14, 22, 23).

In this work, several key gaps were addressed that will enable cell-free production of peptide biologics. First, it is difficult to produce small peptides in CFPS and recombinantly in E. coli due to the presence of proteases (24, 25). Second, the produced therapeutic peptide hormones must also be purified from the background contaminants present in the crude lysate used for CFPS. Fusion proteins are a common method to help improve expression of peptides (26). Therefore, a small ubiquitin-related modifier (SUMO) protein fusion was selected to help stabilize the expressed peptide hormones and to provide a handle for an on-bead, simultaneous enzymatic cleavage and purification system. This process ultimately yields a completely tagless hormone after elution.

### Results and Discussion

A workflow that would allow for point-of-care peptide hormone production by cell-free protein synthesis (CFPS) was developed (FIG. 1a). Using a similar workflow to previous publications (18, 19), lyophilized CFPS reactions, which are stable at ambient temperatures (17), can be prepared, shipped, and stored at the hospital or medical facility where the medication will be needed. They can then be rehydrated as needed with the plasmid DNA that encodes for the needed peptide hormone to start expression. When the CFPS is finished, the hormones are then purified for delivery to patients. To develop this platform, we selected several peptide hormones as model peptides for development (Table 1, below). The peptides were selected both for their use as therapeutics and for theoretical ease of expression in cell-free systems. Therefore, these peptides have minimal or no disulfide bonding, no non-canonical amino acids, and no post-translation modifications. They vary in size from 1 kDa-22 kDa and comprise a variety of different physiologic functions. A leucine zipper heterodimer previously used for assembly of heavy and light chain antibody fragments (27) was also tested to assist assembly of correctly disulfide-bonded endogenous insulin without expression of proinsulin and subsequent cleavage of C-peptide.

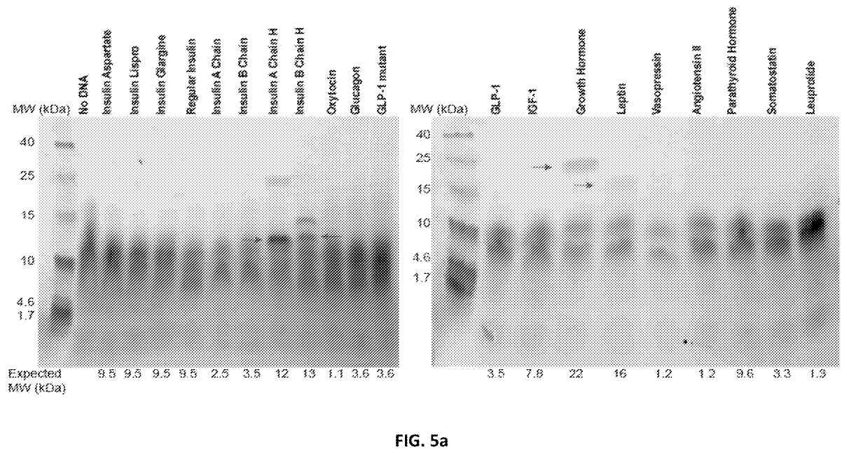

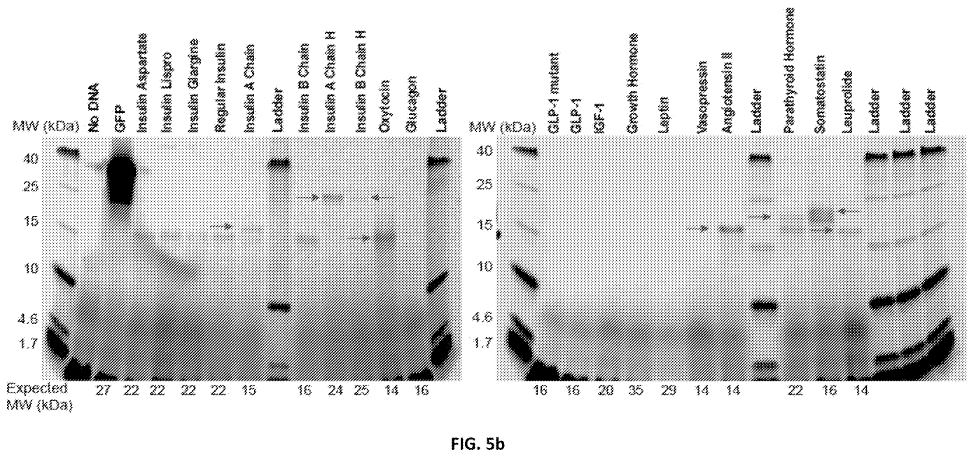

Due to protease degradation in crude extracts, peptide synthesis in CFPS is generally difficult (24, 25). When we attempted to express endogenous hormones in standard BL21 extract, we only observed full length expression of 4 out of the 20 tested constructs (FIG. 5a). Consistent with previous observations, these peptide hormones (insulin A and B chains fused with the leucine zipper heterodimers, growth hormone, and leptin) tended to be the larger (>10 kDa) peptide hormones in the panel that were tested. The larger bands in the insulin A and B chain heterodimer lanes may be due to unintended dimerization or trimerization.

To stabilize the peptides during CFPS, we employed a commonly used fusion protein strategy (28, 29). We decided to use the small ubiquitin-related modifier (SUMO) protein for its ability to improve molar expression of recombinantly expressed proteins (28, 30) and also because it is used as reversible post-translational modification in eukaryotic cells (28, 30-32). Therefore, there is a protease (ubiquitin-like protease-1, Ulp-1) that recognizes the tertiary structure of SUMO and cleaves precisely at the C-terminus of the SUMO protein to leave a scarless protein or peptide product (28, 32). This allows for the purification of a panel of different endogenous peptide hormones without needing to verify that additional amino acids from the proteolytic cleavage of more common proteases like tobacco etch virus (TEV) protease interfere with the biologic activity, pharmacokinetics, or pharmacodynamics of the drug substance. Indeed, the addition of the SUMO tag to the peptide hormones improves the expression of peptide hormones in CFPS using standard crude BL21 lysate (FIG. 5b). With the SUMO fusion, 8 of the 20 peptide hormones in the panel were expressed as full-length constructs (purple arrows), showing a clear improvement over the untagged hormones. There are some contaminating bands below the bands of interest that migrate similarly to SUMO protein (12 kDa) and that may represent truncations.

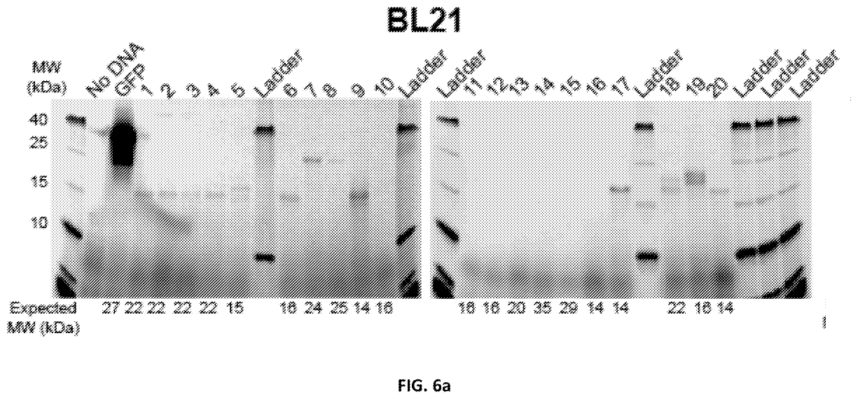

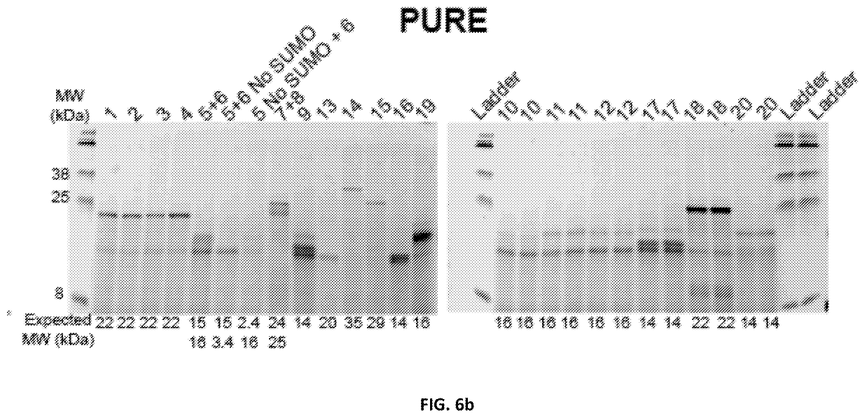

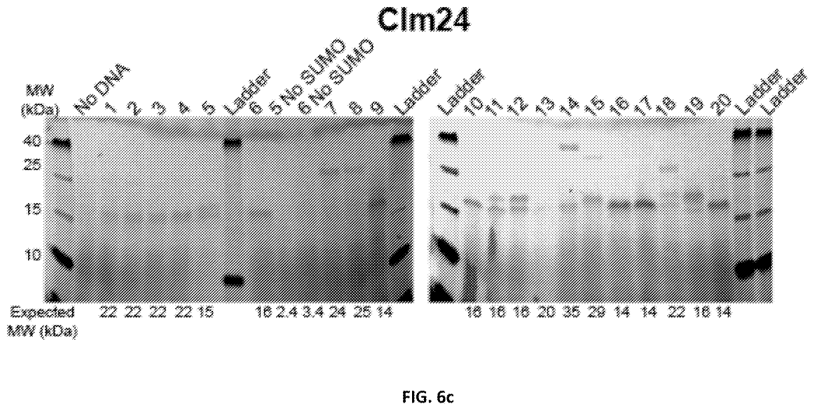

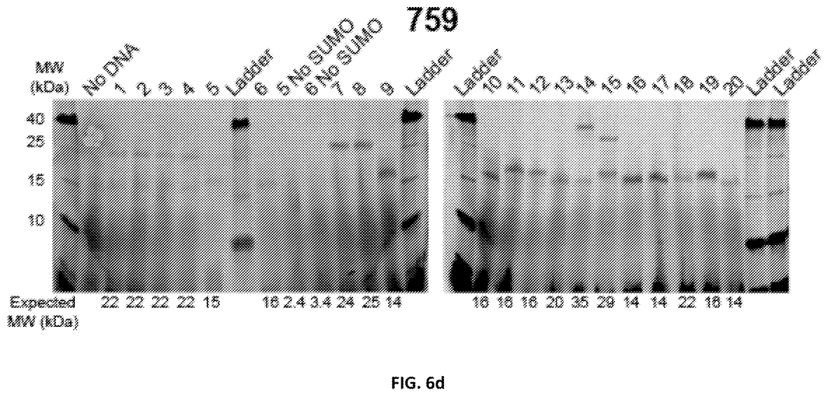

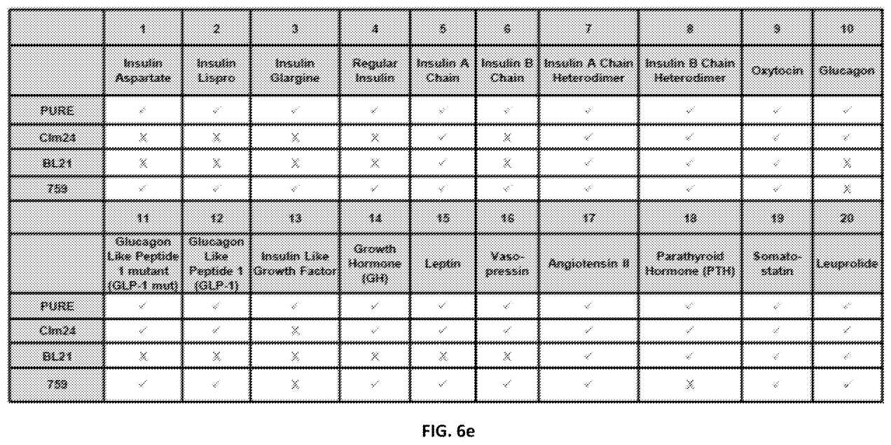

Further optimization was performed to enhance SUMOylated peptide hormone expression by using crude lysates derived from different E. coli strains. The BL21*(DE3) (Fisher Scientific) gel is reprinted from FIG. 5b in FIG. 6a for comparison. All 20 full-length SUMOylated peptide hormones expressed in PUREfrex 2.1 (FIG. 6b), however this PUREfrex 2.1 system in an in vitro transcription and translation system consisting entirely of purified and reconstituted components and therefore is expensive and not as amenable to lyophilization as crude extracts. Clm24 (W3310 Δwaa) (33) is a K strain that was also attempted because there is a version of this strain that has been engineered to have a modified lipopolysaccharide structure thought to reduce its negative endotoxin effects (18), which would further simplify downstream purification and processing of therapeutics generated using this workflow. 14 of the 20 peptide hormones expressed as full-length constructs using Clm24 (FIG. 6c), offering an improvement over the BL21 lysate. Finally, the 759 strain was attempted. This is the 759.T7.Opt strain that was designed by knocking out release factor 1 (RF1) for non-canonical amino acid incorporation, but that also includes a proteolytic-resistant T7 RNAP on the genome that creates some of the highest-yielding extracts reported (34). Of the crude extracts, 759 performed the best as it was able to synthesize 17 out of the 20 peptide hormones (FIG. 6d, e). Therefore, it was selected as the extract of choice for the following experiments.

Peptide hormones can be expressed as soluble biologics in point-of-care enabled CFPS.

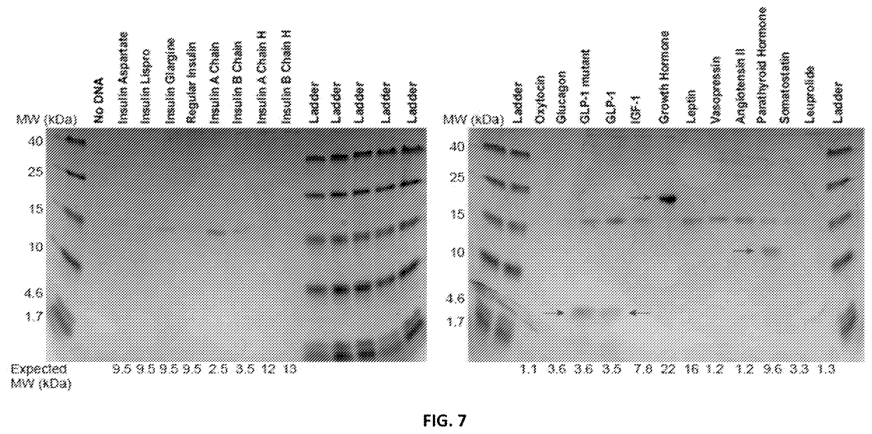

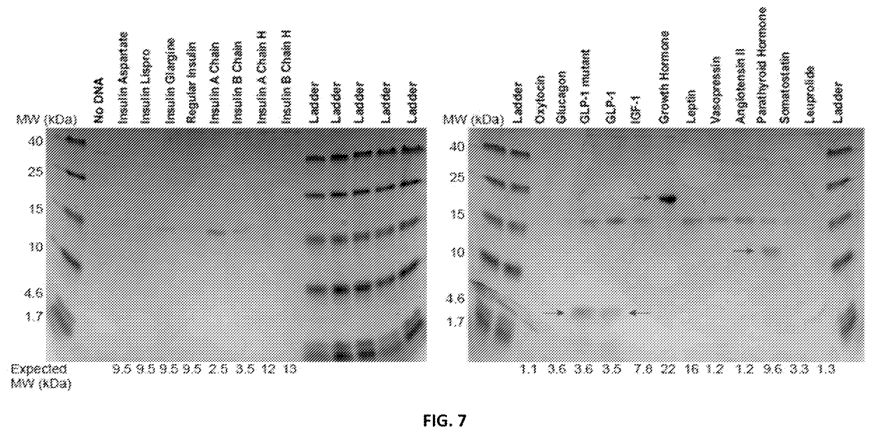

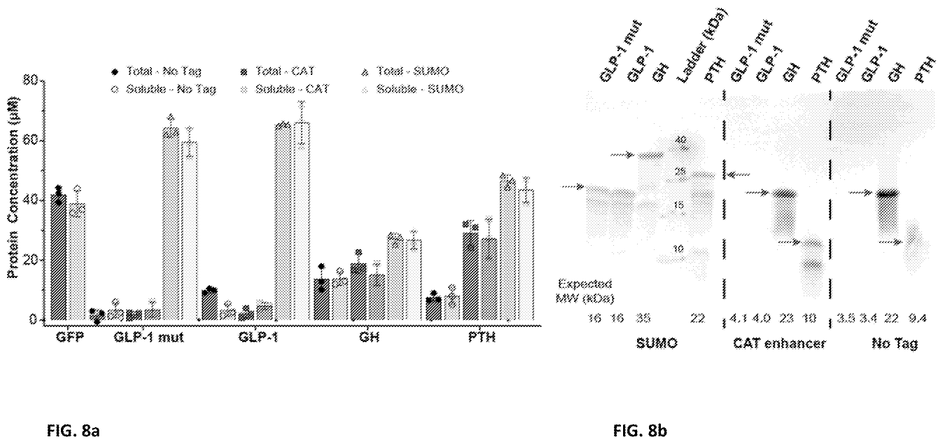

An initial purification screen selected four of the most promising hormones for further study and optimization (FIG. 7). The entire panel of peptide hormones was subjected to affinity purification and cleavage of the SUMO protein to leave untagged peptide hormones. The GLP-1 mutant, GLP-1, growth hormone, and parathyroid hormone were successfully recovered as detected by a Coomassie stained SDS-PAGE gel. These hormones were selected as the focus of the remaining experiments. Recent clinical data has highlighted the promise of using GLP-1 receptor agonists to treat both diabetes and obesity (7, 8). Although the chemical modifications necessary to create GLP-1 receptor agonists that have reasonable therapeutic half-lives are not yet easily accessible in CFPS, there is literature that supports the therapeutic benefit of continuous infusion of GLP-1 in type 1 diabetic patients (35). Although amidation of the C-terminus (7-36) improves half-life, the glycine extension (7-37) has the same biologic effect (36) and is easier to make in CFPS. Therefore, the glycine extension version of GLP-1 was selected for testing. In addition, the GLP-1 mutant was designed to mimic the amino acid changes of liraglutide without the addition of the fatty acyl chain to show robustness of the method to small mutations in the hormone sequence. Growth hormone and parathyroid hormone are also important therapeutic hormones that were recovered in substantial amounts after purification as intact hormones. To quantitatively confirm that each of the four peptide hormones benefit from the SUMO tag, we performed an experiment using incorporation of radioactive ¹⁴C-leucine to determine the expression levels of untagged hormones, hormones with a short CAT enhancer sequence (37), and SUMOylated hormones. There is a clear benefit to including the SUMO fusion, especially for the GLP-1 and GLP-1 mutant (FIG. 8a). With the SUMO fusion, all peptide hormones express at a soluble molar concentration similar to green fluorescent protein (GFP), a positive control protein known to express well in CFPS. An autoradiogram confirms that the SUMOylated hormones express as full-length constructs (purple arrows, FIG. 8b), with smaller bands likely resulting from truncations.

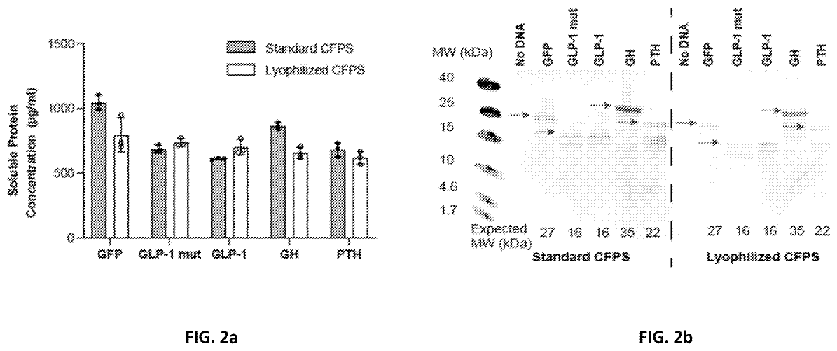

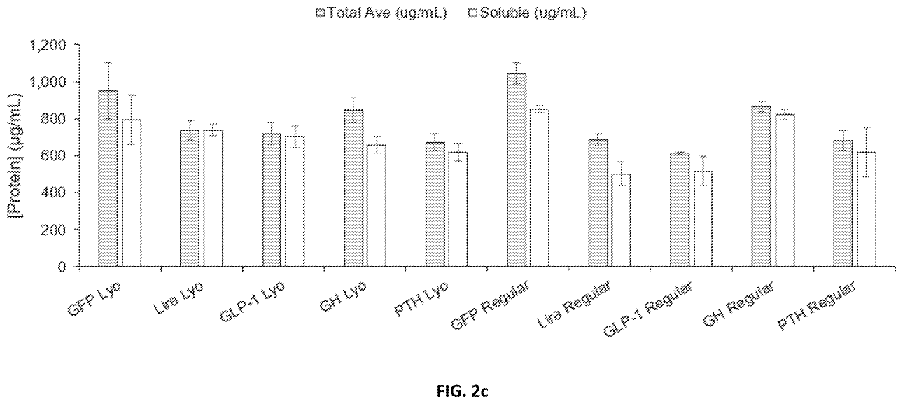

Lyophilization of CFPS reactions is an important component of the point-of-care workflow because it allows CFPS reactions to be stored at room temperature, thus eliminating the need to maintain cold chain during transportation and storage (18, 19). This improves access to sensitive biologic drugs, especially in resource-limited settings and allows for stockpiling in case of emergency need for a biologic drug. We ensured that lyophilization does not significantly impact yields of the SUMO-hormone fusions using radioactive ¹⁴C-leucine incorporation to compare hormones produced in CFPS reactions that had not been lyophilized (“standard”) to those that had undergone lyophilization. Lyophilization does not significantly impact soluble yields of the SUMOylated peptide hormones, and all the hormones express similarly to the positive control GFP (FIG. 2a). An autoradiogram was performed to confirm the full-length constructs were expressed (FIG. 2b). As with previous Fluorotect™ gels (FIG. 6), both full length constructs (purple arrows) and truncations that run at approximately the molecular weight of SUMO without the hormones are observed. There does not appear to be any difference between the standard and lyophilized samples.

A simultaneous 2-in-1 enzymatic purification scheme isolates unmodified peptide hormones.

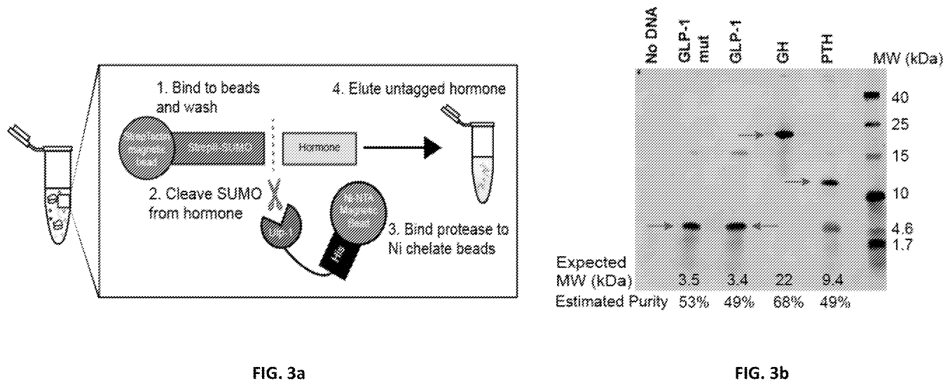

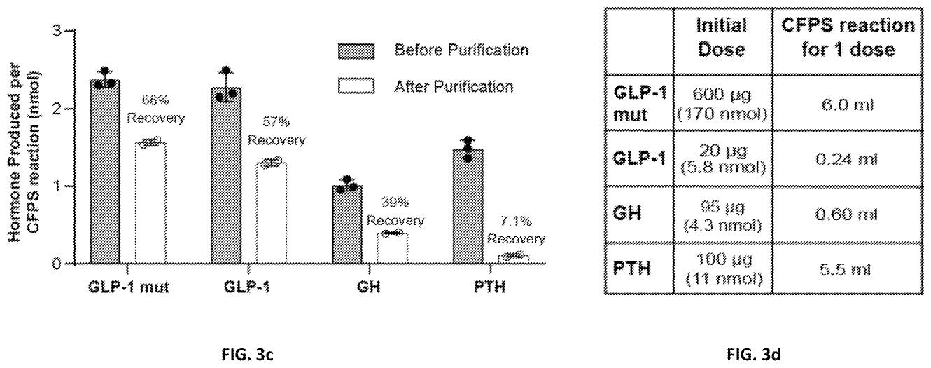

The purification scheme was designed to both purify the peptide hormones from the background crude lysate contaminants and to cleave the SUMO protein off the synthesized hormones to leave endogenous, untagged hormones. The purification scheme involves first binding the N-terminal StrepII-SUMO peptide hormone fusion to Strep-Tactin® functionalized magnetic beads and washing away background CFPS contaminants. Ulp-1 protease then recognizes the tertiary structure of SUMO and cleaves precisely at the SUMO C-terminus to release untagged hormone (32). Because the StrepII tag remains at the N-terminus of SUMO, SUMO remains bound to the magnetic beads. Nickle chelate beads are added to remove the His-tagged Ulp-1 protease from solution and the purified, untagged peptide hormone is collected (FIG. 3a). Previous work has been done where enzymatic cleavage reactions were used to elute protein off purification resin with a TEV protease variant (38), a picornavirus 3C protease (39), a PreScission™ protease (40), and even with scSUMO (41). However, to our knowledge, this work represents the first effort to use SUMO to both stabilize the expression of therapeutics and to provide a handle for purification of an untagged version of the peptide or protein.

This scheme functioned to purify the peptide hormones to approximately 50% purity by densitometry on a Coomassie stained SDS-PAGE gel (FIG. 3b). The purity was calculating by taking the ratio of the intensity of the band of interest to the intensity of the entire lane. There are relatively clean bands (purple arrows) with minor contaminants in all the lanes containing peptide hormones. Because of the nature of the Ulp-1 protease cleavage site directly after the last two glycine residues at the C-terminus, these bands represent endogenous hormones with a precisely defined amino acid sequence lacking any scar sequence or remaining amino acids resulting from the original fusion with SUMO.

The amount of hormone recovered was calculated by using a bicinchoninic acid (BCA) assay to quantitate total protein in the purified samples and then using purity from the densitometry analysis in FIG. 3b to calculate the final peptide hormone concentration. When compared to the amount of protein expressed in the CFPS reaction (FIG. 2a), the recovery after purification ranges from 7.1% to 66% (FIG. 3c). This yield is low compared to typical affinity purification schemes; however, it theoretically could be improved by optimizing SUMO cleavage conditions or changing the method of affinity purification from a magnetic bead scheme to a set up with resin in a column.

CFPS reactions have been shown to scale up to 100 L (42), so we used the purification yields in FIG. 3c to calculate the size of CFPS reactions that would be necessary to produce a therapeutic dose assuming the expression and purification yields are maintained during scale up and summarized this information in FIG. 3d. Since the GLP-1 mutant is designed based on liraglutide, and a typical liraglutide dose is 600 μg (43), it would take 6.0 ml (109 55 μl reactions that produce 5.5 ug (1.57 nmol) each) of CFPS to produce enough GLP-1 mutant for 1 dose. Typically, GLP-1 receptor agonists have modifications that improve half-life; however, GLP-1 has been used in research to treat patients with type 1 diabetes using a continuous infusion of 1.2 pmol/kg/min of GLP-1 throughout a meal (35). Assuming an 80 kg adult that eats for 60 minutes, this would correspond to a total of 20 μg (5.8 nmol). To produce this amount of GLP-1, it would take a 240 μl (4.39 55 μl reactions that each produce 4.6 μg (1.31 nmol)) CFPS reaction to make enough GLP-1 for one dose. Growth hormone is typically dosed by weight and 0.33 mg/kg/week is a normal starting dose (10). For an average 20 kg 5-year-old boy, the daily dose would be 95 μg. 600 μl (10.79 55 μl reactions that each produce 8.8 μg (0.4 nmol)) of CFPS would be required to produce enough GH for one dose. To treat osteoporosis, a typical initial dose of parathyroid hormone is 100 μg (44). 5.5 ml (100 55 μl reactions that each produce 1 μg (0.1 nmol)) would be necessary to produce enough PTH for one dose. The CFPS reaction volumes required for one dose of any of those therapeutics is well within the range of feasibility.

Characterization by LC-MS, ELISA, AlphaLISA, and BLI shows cell-free produced growth hormone is intact and capable of binding its receptor.

The activity and function of the purified proteins are important to verify for future therapeutic uses. We sought to confirm the cleaved hormones are the appropriate size by coupled liquid chromatography-mass spectrometry (LC-MS), ensure recognition of the hormones by two orthogonal antibodies in a sandwich enzyme-linked immunosorbent assay (ELISA), and test hormone binding to its respective receptor with both an amplified luminescent proximity homogeneous assay (AlphaLISA) and biolayer interferometry (BLI).

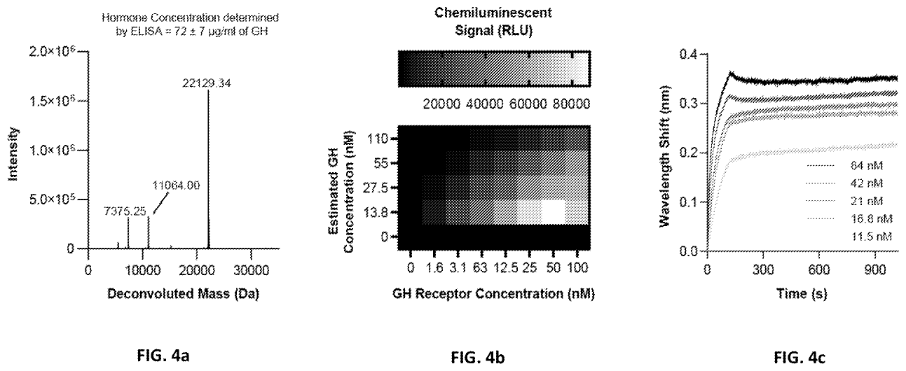

We first showed this for growth hormone (FIG. 4). Protein LC-MS after treatment of the sample with dithiothreitol (DTT) revealed an observed mass of 22129.34 Da from maximum entropy deconvolution which matches the theoretical GH mass (22129.05 Da) with an error of 13 ppm (FIG. 4a). GH was also recognized by two orthogonal antibodies in a commercial sandwich ELISA and showed a recovery of 72±7 μg/ml (FIG. 4a). This is approximately 45% of the amount detected by BCA and densitometry. We also assessed GH engagement with the extracellular domain of its receptor with AlphaLISA (FIG. 4b). AlphaLISA is an assay that can detect protein-protein interactions using proteins immobilized on two separate beads. One protein is immobilized on donor beads that are excited by a laser to release a singlet oxygen species that can diffuse up to approximately 100 nm in solution. If the beads are in close proximity due to the interaction of the two species, the singlet oxygen will diffuse to the acceptor bead and trigger another chemical reaction to release a chemiluminescent signal that can be detected by a plate reader (45). In this case, we added a FLAG tag evolved to have a higher affinity for anti-FLAG antibodies (sFLAG) to the C-terminus of the peptide hormones (46) and then used anti-FLAG donor beads in the assay. The commercially produced extracellular domain of the growth hormone receptor (GHR, Acro GHR-H52222) was His-tagged and immobilized on nickel chelate acceptor beads. A cross-titration of both growth hormone and its receptor revealed a pattern consistent with clear binding, suggesting that the GH produced in CFPS and purified using our workflow can recognize and engage with its receptor. The binding was confirmed with BLI, a separate, orthogonal binding method. BLI can detect the tagless hormones interacting with their respective receptors because it measures the wavelength shift of reflected light due to protein or peptide accumulation on the sensor tip. A range of estimated GH concentrations calculated using the BCA and densitometry method from 11.5-84 nM were tested. Association, where the tip that was coated with the extracellular domain of GHR using the nickel chelate-his tag interaction was dipped in a solution of peptide hormone, was assessed from 0-120 s. Dissociation, where the sensor tip was then moved to buffer that did not contain any GH, was assessed from 120-1020 s. A K_{D} value for the hormone-receptor binding was not able to be determined because there was no significant dissociation observed, so the algorithm could not fit the data accurately. However, clear binding was observed (FIG. 4c), which confirms the binding observed by AlphaLISA in FIG. 4b.

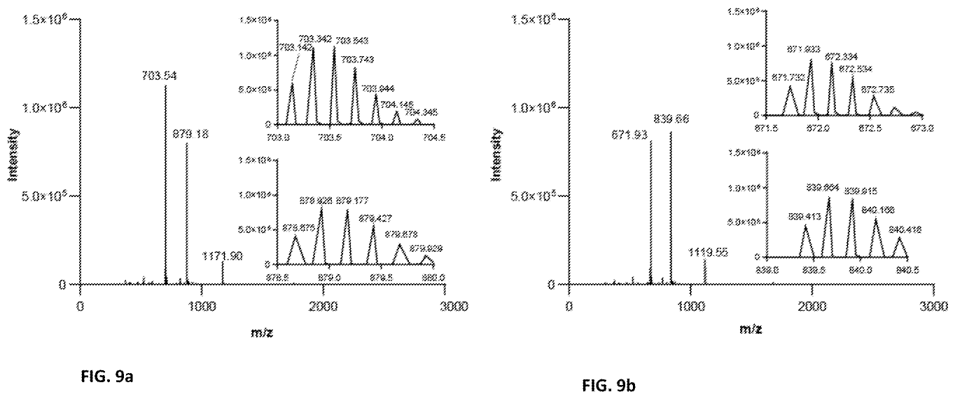

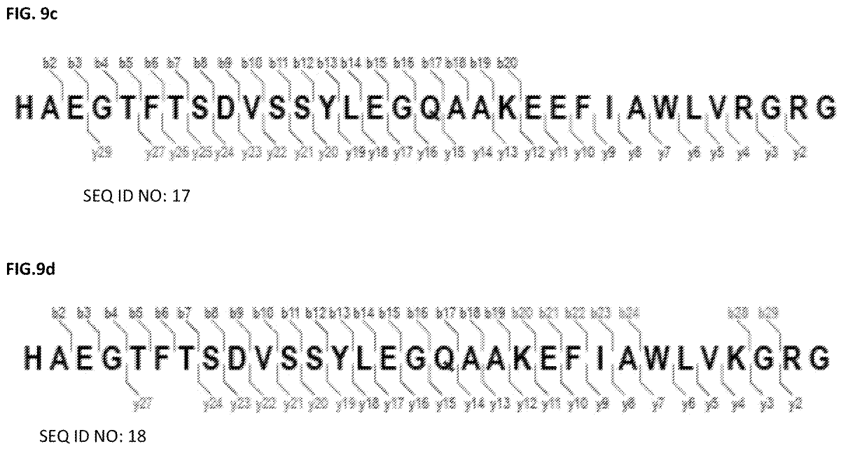

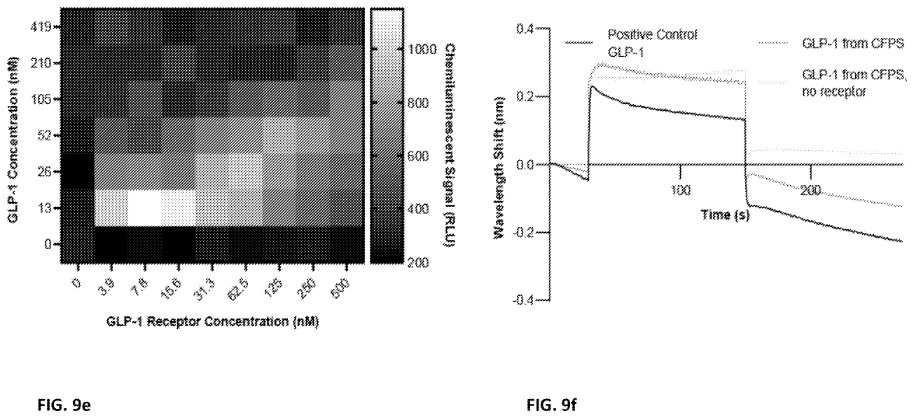

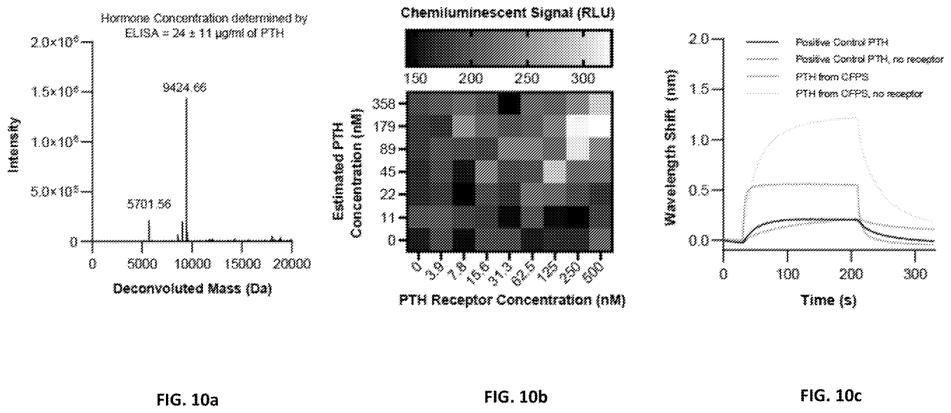

Similar experiments to characterize the produced peptides were run for GLP-1 (FIG. 9) and parathyroid hormone (FIG. 10). GLP-1 and the GLP-1 mutant were detected as full-length peptides in peptide LC-MS and their sequences were confirmed with MS/MS (FIG. 9a-d). The observed and theoretical monoisotopic masses of the GLP-1 mutant were 3510.67 Da and 3510.72 Da, respectively, which gives an error of 15 ppm (FIG. 9a). The observed and theoretical monoisotopic masses of GLP-1 were 3353.62 Da and 3353.67 Da, respectively, which gives an error of 15 ppm (FIG. 9b). MS/MS analyses of the ions corresponding to both the GLP-1 mutant (FIG. 9c) and GLP-1 (FIG. 9d) had fragmentation patterns consistent with the theoretical b and y ion fragmentation pattern. In fact, the entire sequences except for the two N-terminal and C-terminal amino acids were confirmed for both GLP-1 mutant and GLP-1 in these experiments. The peptide hormones are cleaved precisely at the C-terminus of SUMO to give tagless hormones as expected. Using sFLAG tagged GLP-1, anti-FLAG donor beads, Fc-tagged extracellular domain of the GLP-1 receptor, and Protein A acceptor beads, AlphaLISA detected GLP-1 binding to its receptor (FIG. 9e). There was low signal relative to GH, however, the binding signal is still more than 3 standard deviations above the background noise. Unfortunately, this binding was unable to be confirmed with BLI, likely because the hormone is too small (3.5 kDa) to generate enough of a wavelength shift to be detected by the instrument (FIG. 9f). There was no binding detected at a level greater than the negative control (GLP-1 without receptor immobilized on the sensor tip, light orange) for either the GLP-1 positive control (commercially purchased GLP-1, PeproTech 130-08, black) or the GLP-1 produced in CFPS (dark orange). Full-length parathyroid hormone was detected in the deconvoluted LC-MS spectra with an observed mass of 9424.66 Da, which matches the theoretical mass of 9424.73 Da with an error of 7.4 ppm (FIG. 10a). PTH was also detected by two orthogonal antibodies in a commercial sandwich ELISA assay (Biomatik EKF58372). The concentration was determined to be 24±11 μg/ml with 4 dilutions of a single sample (FIG. 10a). This value is approximately 80% of that detected by BCA and densitometry. AlphaLISA suggested that PTH interacts with the extracellular domain of its receptor (FIG. 10b). Using sFLAG-tagged PTH, anti-FLAG donor beads, His-tagged PTH receptor (Abcam ab 182670), and nickel chelate acceptor beads, there is a very low signal that has a typical binding pattern. Unfortunately, due to high background binding and a relatively small PTH (9.5 kDa), BLI was also unable to detect PTH binding to the extracellular domain of its receptor (FIG. 10c). Black lines represent a commercially purchased positive control PTH (OriGene SA6052) where there is no receptor immobilized on the lightly shaded line and receptor immobilized with the dark line. Orange lines represent PTH produced in CFPS, again where there is no receptor immobilized on the tip for the lightly shaded line and receptor immobilized with the dark line. In both samples, the high background binding is obscuring any possible true binding signal from the hormone specifically binding its receptor, so we were unable to confirm binding with BLI.

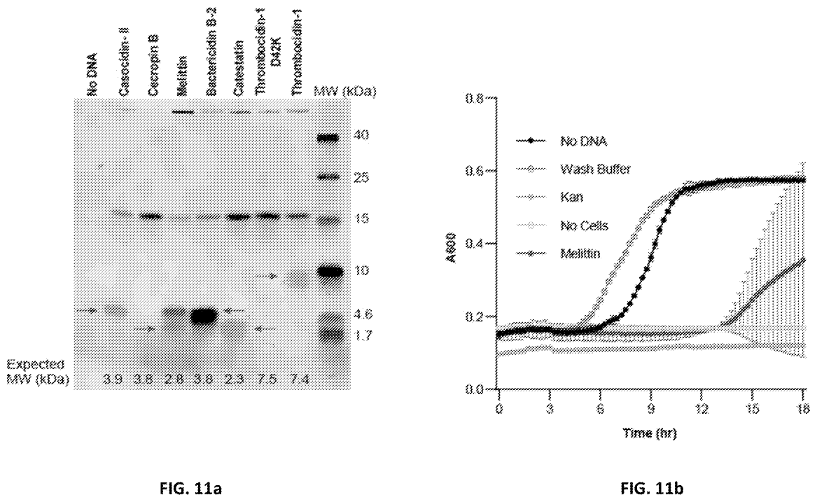

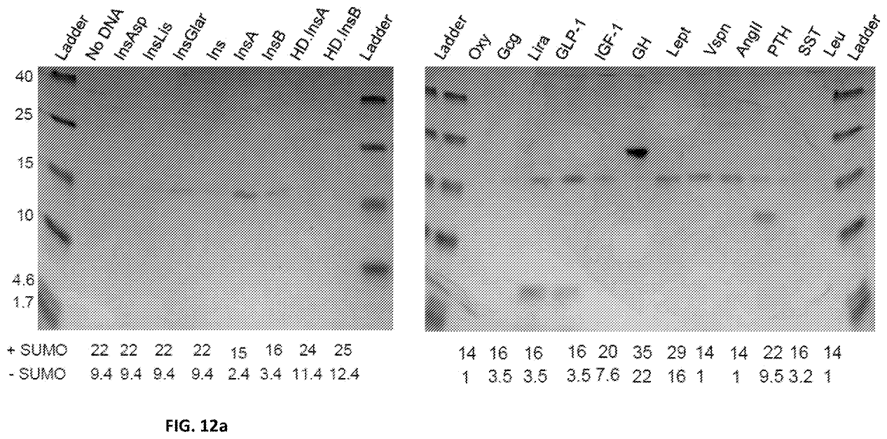

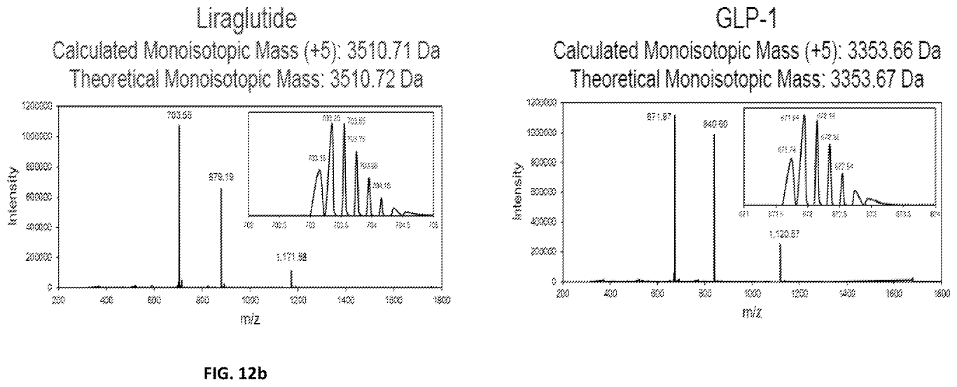

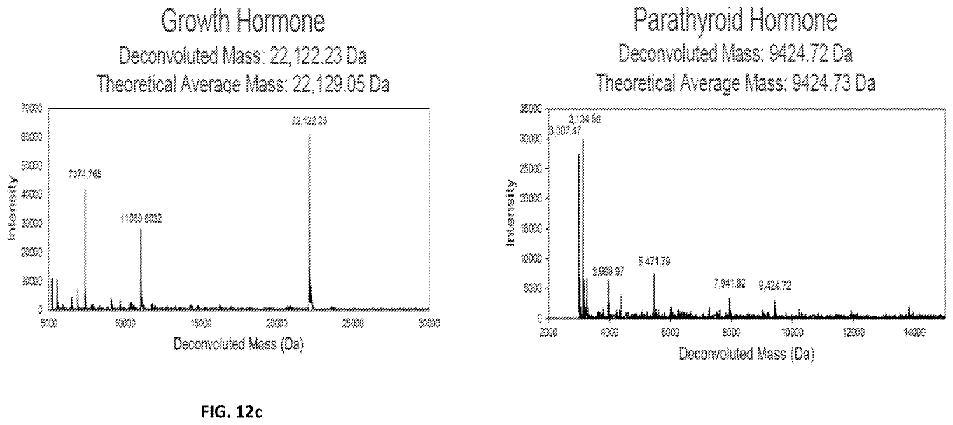

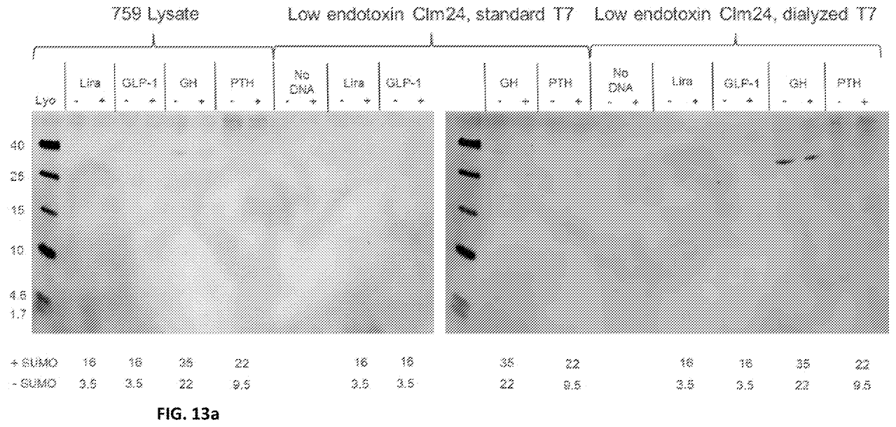

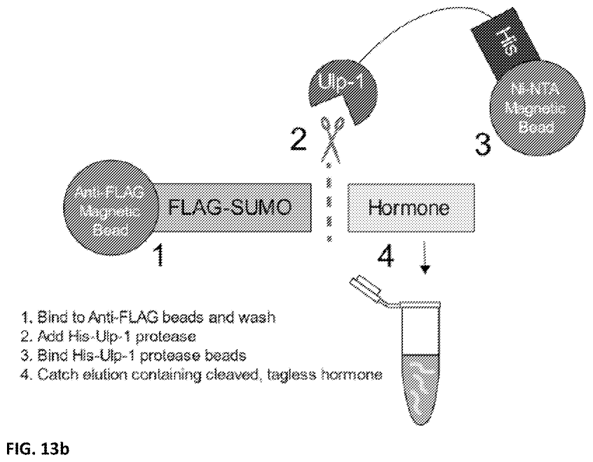

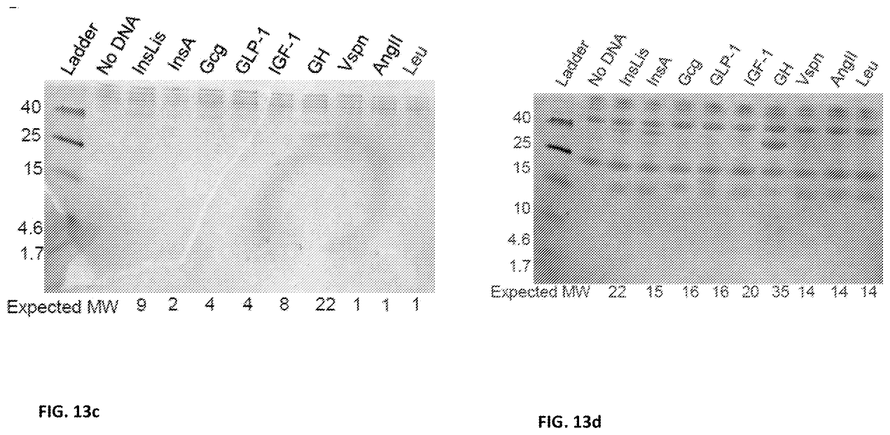

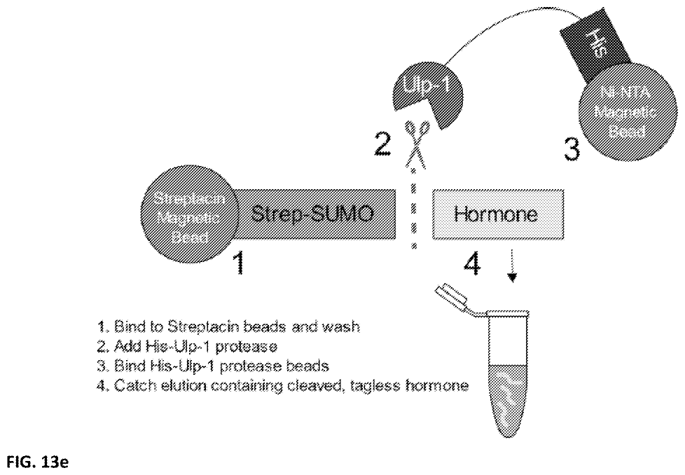

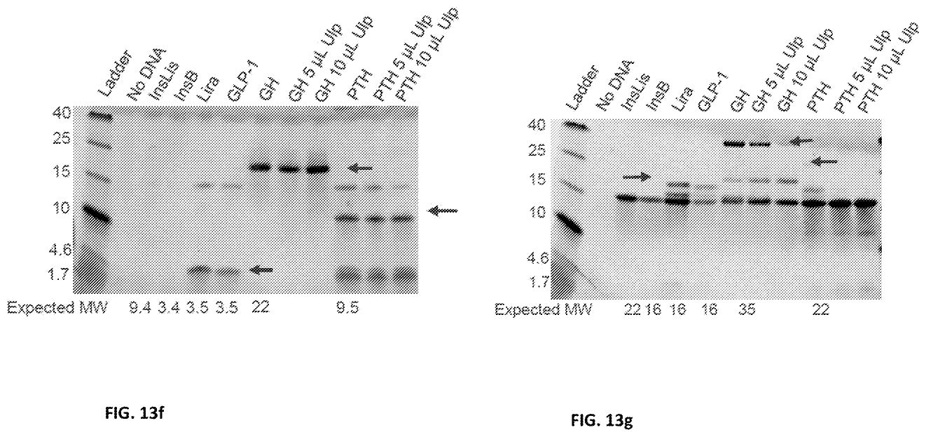

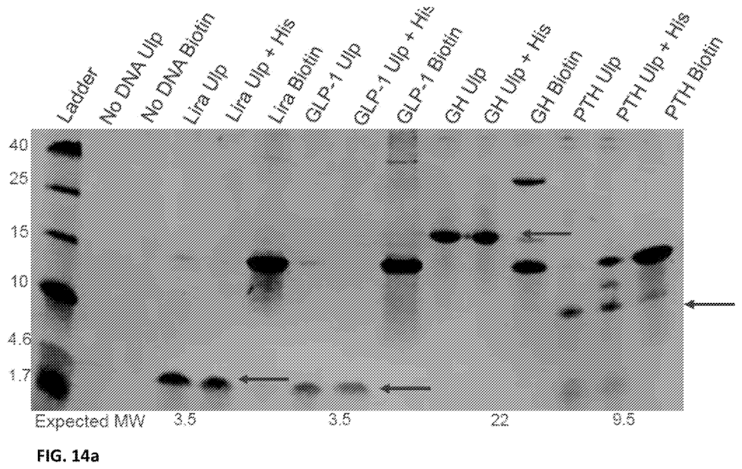

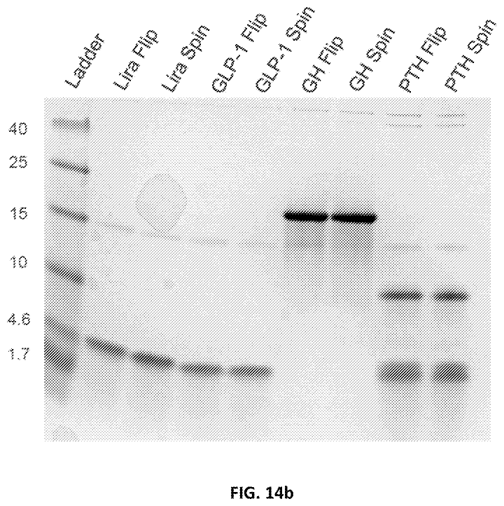

We then wanted to extend this expression and purification method to other peptides and decided to try a panel of antimicrobial peptides. After the full expression and purification scheme, we were able to recover five peptides detected by Coomassie stained SDS-PAGE-Casocidin-II, Melittin, Bactericidin B-2, Catestatin, Thrombocidin-1 D42K, and Thrombocidin-1 (purple arrows, FIG. 11a). In an overnight incubation of E. coli strain MG1655 in a 384 well plate in two replicates, melittin was shown to be functional by inhibiting growth of the bacteria (FIG. 11b), showing that this workflow can be used in applications outside of peptide hormones.

### CONCLUSIONS

Overall, we have created an expression and first-pass purification procedure for peptide hormones that could be integrated into the suitcase platform designed by Adiga and coworkers (21) to be performed at the point-of-care. Further development efforts are necessary to ensure that the hormones would be safe for injection into patients, particularly with regards to the purity of the final product. Endotoxin removal and polishing purification steps would need to be integrated into the system. However, this work represents a step towards the ability to reproducibly synthesize biologics, including peptide hormones, at the point-of-care in resource-limited settings. This work also expanded the synthesis capabilities of CFPS for therapeutically relevant molecules, particularly for biologics with molecular weights smaller than 10 kDa.

### Materials and Methods

### Genes

All constructs were expressed in the pJL1 plasmid (Addgene 69496). A CAT enhancer sequence (MEKKI) (SEQ ID NO: 1) was present at the N-terminus, followed by a linker (RGS) to the Strep-II tag (WSHPQFEKTG) (SEQ ID NO:2). The SUMO protein follows without a linker and then the peptide hormone is at the C-terminus of the SUMO protein. For antimicrobial peptides, the gene is in the same location as the peptide hormone. The amino acid sequences for these constructs are given below. For sFLAG constructs designed for AlphaLISA experiments, a GS7 linker (GGGSGGG) (SEQ ID NO: 3) separates the sFLAG amino acid sequence evolved for higher affinity to anti-FLAG antibodies (DYKDEDLL) (SEQ ID NO: 4) (46) from the C-terminus of the hormone. The heterodimer (27) is located after a GSSGS (SEQ ID NO: 5) linker at the C-terminus of the affinity tag.

SUMO (SEQ ID NO: 6):
MASGSDSEVNQEAKPEVKPEVKPETHINLKVSDGSSEIFFKIKKTTPLRR
LMEAFAKRQGKEMDSLRFLYDGIRIQADQTPEDLDMEDNDIIEAHREQIG
G
Insulin Aspartate (SEQ ID NO: 7):
FVNQHLCGSHLVEALYLVCGERGFFYTDKTRREAEDLQVGQVELGGGPGA
GSLQPLALEGSLQKRGIVEQCCTSICSLYQLENYCN
Insulin Lispro (SEQ ID NO: 8):
FVNQHLCGSHLVEALYLVCGERGFFYTKPTRREAEDLQVGQVELGGGPGA
GSLQPLALEGSLQKRGIVEQCCTSICSLYQLENYCN
Insulin Glargine (SEQ ID NO: 9):
FVNQHLCGSHLVEALYLVCGERGFFYTPKTRREAEDLQVGQVELGGGPGA
GSLQPLALEGSLQKRGIVEQCCTSICSLYQLENYCG
Regular Insulin (SEQ ID NO: 10):
FVNQHLCGSHLVEALYLVCGERGFFYTPKTRREAEDLQVGQVELGGGPGA
GSLQPLALEGSLQKRGIVEQCCTSICSLYQLENYCN
Insulin A Chain (SEQ ID NO: 11):
GIVEQCCTSICSLYQLENYCN
Insulin B Chain (SEQ ID NO: 12):
FVNQHLCGSHLVEALYLVCGERGFFYTPKT
A chain heterodimer (SEQ ID NO: 13):
GTKEDILERQRKIIERAQEIHRRQQEILEELERIIRKPGSSEEAMKRMLK
LLEESLRLLKELLELSEESAQLLYEQR
B chain heterodimer (SEQ ID NO: 14):
GTEKRLLEEAERAHREQKEIIKKAQELHRRLEEIVRQSGSSEEAKKEAKK
ILEEIRELSKRSLELLREILYLSQEQKGSLVPR
Oxytocin (SEQ ID NO: 15):
CYIQNCPLG
Glucagon (SEQ ID NO: 16):
HSQGTFTSDYSKYLDSRRAQDFVQWLMNT
GLP-1 mut (SEQ ID NO: 17):
HAEGTFTSDVSSYLEGQAAKEEFIAWLVRGRG
GLP-1 (SEQ ID NO: 18):
HAEGTFTSDVSSYLEGQAAKEFIAWLVKGRG
IGF-1 (SEQ ID NO: 19):
GPETLCGAELVDALQFVCGDRGFYFNKPTGYGSSSRRAPQTGIVDECCFR
SCDLRRLEMYCAPLKPAKSA
Growth Hormone (SEQ ID NO: 20):
FPTIPLSRLFDNAMLRAHRLHQLAFDTYQEFEEAYIPKEQKYSFLQNPQT
SLCFSESIPTPSNREETQQKSNLELLRISLLLIQSWLEPVQFLRSVFANS
LVYGASDSNVYDLLKDLEEGIQTLMGRLEDGSPRTGQIFKQTYSKFDTNS
HNDDALLKNYGLLYCFRKDMDKVETFLRIVQCRSVEGSCGF
Leptin (SEQ ID NO: 21):
VPIQKVQDDTKTLIKTIVTRINDISHTQSVSSKQKVTGLDFIPGLHPILT
LSKMDQTLAVYQQILTSMPSRNVIQISNDLENLRDLLHVLAFSKSCHLPW
ASGLETLDSLGGVLEASGYSTEVVALSRLQGSLQDMLWQLDLSPGC
Vasopressin (SEQ ID NO: 22):
CYFQNCPRG
Angiotensin II (SEQ ID NO: 23):
DRVYIHPF
Parathyroid Hormone (SEQ ID NO: 24):
SVSEIQLMHNLGKHLNSMERVEWLRKKLQDVHNFVALGAPLAPRDAGSQR
PRKKEDNVLVESHEKSLGEADKADVNVLTKAKSQ
Somatostatin (SEQ ID NO: 25):
SANSNPAMAPRERKAGCKNFFWKTFTSC
Leuprolide (SEQ ID NO: 26):
QHWSYGLRPG
Casocidin-II (SEQ ID NO: 27):
TKLTEEEKNRLNFLKKISQRYQKFALPQYLK
Cecropin B (SEQ ID NO: 28):
KWKIFKKIEKVGRNIRNGIIKAGPAVAVLGEAKAL
Melittin (SEQ ID NO: 29):
GIGAVLKVLTTGLPALISWIKRKRQQ
Bactericidin B-2 (SEQ ID NO: 30):
WNPFKELERAGQRVRDAVISAAPAVATVGQAAAIARG
Catestatin (SEQ ID NO: 31):
SSMKLSFRARAYGFRGPGPQL
Thrombocidin-1 D42K (SEQ ID NO: 32):
AELRCMCIKTTSGIHPKNIQSLEVIGKGTHCNQVEVIATLKKGRKICLDP
DAPRIKKIVQKKLAGDES
Thrombocidin-1 (SEQ ID NO: 33):
AELRCMCIKTTSGIHPKNIQSLEVIGKGTHCNQVEVIATLKDGRKICLDP
DAPRIKKIVQKKLAGDES
Preparation of cell extracts for CFPS

Crude extracts for CFPS were made from several Escherichia coli strains. The “CLM24” strain is the W3110 strain with a knockout of the ligase that transfers the O polysaccharide to the lipid-A core (WaaL), engineered for N-linked protein glycosylation (33). The “BL21” strain is the BL21 Star™ (DE3) (Fisher Scientific) strain, and the “759” strain has a release factor 1 (RF1) knockout from the C321.ΔA strain with integration of T7 RNA polymerase with mutations K183G and K190L to prevent OmpT cleavage onto the genome (759.T7.Opt) (34). Cells were grown in 2×YTP media (5 g/L sodium chloride, 16 g/L tryptone, 10 g/L yeast extract, 7 g/L potassium phosphate monobasic, and 3 g/L potassium phosphate dibasic, pH 7.2) in 1 L shake flasks or in a Sartorius Stedim BIOSTAT Cplus 10 L bioreactor. BL21 and CLM24 were grown at 37° C. and 759 was grown at 34° C. At an OD₆₀₀ of ˜ 0.6, T7 RNA polymerase expression was induced with 1 mM IPTG in the BL21 and 759 strains. At an OD₆₀₀ of ˜2.8, cells were harvested and pelleted by centrifugation at 8000×g for 5 min at 4° C. Cells were washed with chilled S30 buffer (10 mM Tris-acetate pH 8.2, 14 mM magnesium acetate, 60 mM potassium acetate) 3 times and pelleted by centrifugation at 10000×g for 2 min at 4° C. 1 mL/g wet cell mass of S30 buffer was used to resuspend BL21 and CLM24 cells and 0.8 ml/g wet cell mass of S30 buffer was used to resuspend 759 cells. 1 ml aliquots of BL21 and 1.4 ml aliquots of 759 cells were lysed by a Q125 Sonicator (Qsonica) at 50% amplitude. BL21 cells were pulsed 10 s on, 10 s off until 640 J were reached, and 759 cells were pulsed for 45 s on and 59 s off until 950 J were reached. CLM24 cells were lysed with single pass through an Avestin EmulsiFlex-B15 at 20,000 to 25,000 psig. The lysates were centrifuged for 10 min at 4° C. BL21 was spun at 10000×g and 759 and CLM24 were spun at 12000×g. The supernatant was collected and BL21 was aliquoted and flash frozen. 759 and CLM24 both underwent runoff reactions for 1 hr at 37° C. with 250 rpm shaking and were spun at 12000×g and 10000×g, respectively, for 10 min at 4° C. Supernatants were collected and samples were aliquoted, flash frozen, and stored at −80° C. until use.

### Cell-Free Protein Synthesis (CFPS)

55 μl CFPS reactions were carried out in 2 ml microcentrifuge tubes (Axygen) using a modified PANOx-SP system (47). 12 mM magnesium glutamate, 10 mM ammonium glutamate, 130 mM potassium glutamate, 0.85 mM each of GTP, UTP, and CTP, 1.2 mM ATP, 34 μg/ml folinic acid, 0.171 mg/ml E. coli tRNA, 0.33 mM NAD, 0.27 mM CoA, 4 mM oxalic acid, 1 mM putrescine, 1.5 mM spermidine, 57 mM HEPES, 2 mM 20 amino acids, 0.03 M phosphoenolpyruvate, 13.3 mg/ml plasmid purified using Zymo Midiprep Kits. If lyophilized, 30 mg/ml of sucrose was added to the reaction as a lyoprotectant. 45.8 μl of pre-mix with all components except for plasmid were dried for at least 4 hours after flash freezing on a VirTis BenchTop Pro lyophilizer (SP Scientific) at 100 mtorr and −80° C. Lyophilized pre-mix was rehydrated with 45.8 μl of nuclease free water (Fisher Scientific AM9937) and then added to tubes containing 9.2 μl of 80 mg/ml of plasmid DNA. Reactions were incubated overnight at 30° C.

### Radioactive Quantification of CFPS Yields

Soluble CFPS yields were quantified by incorporation of 1+C-Leucine (PerkinElmer) as previously described (48). After lyophilization, reactions were rehydrated with ¹⁴C-Leucine in water to give a final 10 μM concentration in triplicate 15 μl reactions. Untagged hormones were not lyophilized. These reactions were transferred to tubes containing the appropriate plasmid and incubated overnight at 30° C. 6 μl of soluble fractions were taken by removing the supernatant of reactions spun at 16.1 k×g for 10 min at 4° C. and amino acids were cleaved off charged tRNA by incubation at 37° C. for 20 min with 0.25 N KOH. 5 μl of solutions were spotted on a 96 well filtermat (PerkinElmer 1450-421), dried, then precipitated by a 5% solution of trichloroacetic acid at 4° C. The filtermat was dried and radioactivity was measured by a liquid scintillation counter (PerkinElmer MicroBeta) compared to a filtermat with the same solutions that had not been precipitated by trichloroacetic acid.

### Autoradiograms

Samples that were quantified by radioactive 1+C-Leucine incorporation were run on an SDS-PAGE gel to confirm full-length expression. Samples were prepared for SDS-PAGE by adding 5 μl of sample to 4 μl of tricine sample buffer (Bio-Rad 1610739) and 1 μl of 1 M DTT and heat denaturing at 90° C. for 10 min. The samples were run on an 16.5% MINIPROTEAN® Tris-Tricine gel (Bio-Rad 4563066) for 100 V for 90 min at constant voltage. The gels were dried (Hoefer GD2000) and exposed for at least 4 days before imaging on a Typhoon FLA 700 (GE Healthcare).

### Hormone Purification

After overnight CFPS expression, 35 μl of IBA MagStrep “type3” XT Strep-Tactin beads were equilibrated in a wash buffer of tris-buffered saline solution with 0.05% Tween 20 (ThermoFisher 28360) by washing twice with 35 μl of the wash buffer. Wash steps involve separating the magnetic beads from solution by using a magnetic plate and removing the supernatant before adding fresh wash buffer and resuspending the magnetic beads. 55 μl of wash buffer was then added to each CFPS sample and the solution was added to the magnetic beads and spun at ˜18 rpm on a rotator at 4° C. for at least 45 minutes. The beads were then washed 3 times with 120 μl of wash buffer and resuspended in 30 μl of wash buffer. 5 μl of 1 U/μl of Ulp-protease (SigmaAldrich SAE0067) was added to each sample and incubated at room temperature with spinning at ˜18 rpm for at least 2 hours. 2 μl of Dynabeads™ His-Tag Isolation and Pulldown beads (ThermoFisher 10104D) for each CFPS reaction were equilibrated twice in equal volumes of wash buffer before addition to the purified reactions to remove the Ulp-protease and incubated with spinning at 4° C. for at least 30 minutes. The purified solution was removed from the magnetic beads and placed in fresh tubes for further analysis.

SDS-PAGE with Coomassie

Electrophoresis was used to separate peptides in a 16.5% Mini-PROTEAN® Tris-Tricine Gel (Bio-Rad) with tris-tricine (Bio-Rad) or 4× Protein Sample Loading Buffer (LiCOR) and DTT. Tris-tricine running buffer (Bio-Rad 1610744) was used to run the gel at 100 V for 90 minutes. The gel was stained for at least an hour with Bulldog Bio 1-Step Coomassie stain (AS001000) and destained overnight in water.

### Densitometry

A built-in densitometry software (Image Studio Lite) was used to compare the brightness of the band of interest to the entire lane. The ratio of these values was used to determine the purity of the sample.

### BCA

Total protein concentration in the purified samples was determined with the Pierce Rapid Gold BCA Protein Assay kit (ThermoFisher A53225) by comparing absorbance signal at 480 nm to a BSA standard curve ranging from 0-2000 μg/ml using manufacturer's instructions. Hormone samples were run in duplicate with a 10 μl sample volume and 200 μl of working reagent in clear flat-bottomed 96 well plates (Corning 3370).

### Protein LC-MS

Intact hormone molecular weight of large (>6 kDa) hormones was determined by LC-MS by injection of 5 μl of the purified peptide hormone from 3 separate reactions. After surfactant removal with the acidic protocol of the ProteoSpin detergent removal and sample concentration kit (Norgen Biotek 22800), samples were injected into a Bruker Elute UPLC equipped with an ACQUITY UPLC Peptide BEH C4 Column, 300 Å, 1.7 μm, 2.1 mm×50 mm (186004495 Waters Corp.) with a 10 mm guard column of identical packing (186004495 Waters Corp.) coupled to an Impact-II UHR TOF Mass Spectrometer (Bruker Daltonics, Inc.). Liquid chromatography was performed using 0.1% formic acid in water as Solvent A and 0.1% formic acid in acetonitrile as Solvent B at a flow rate of 0.5 mL/min and a 50° C. column temperature. An initial condition of 20% B was held for 1 min before elution of the proteins of interest during a 4 min gradient from 20 to 50% B. The column was washed and equilibrated by 0.5 min at 71.4% B, 0.1 min gradient to 100% B, 2 min wash at 100% B, 0.1 min gradient to 20% B, and then a 2.2 min hold at 20% B, giving a total 10 min run time. An MS scan range of 100-3000 m/z with a spectral rate of 2 Hz was used. External calibration was performed. Data was analyzed using the maximum entropy deconvolution built-in to the Bruker DataAnalysis software.

### Peptide LC-MS

Peptide hormone molecular weight of small (<6 kDa) hormones was determined by LC-MS by injection of 5 μl of the purified peptide hormone from 3 separate reactions. After surfactant removal with the acidic protocol of the ProteoSpin detergent removal and sample concentration kit (Norgen Biotek 22800), samples were injected into a Bruker Elute UPLC equipped with an ACQUITY UPLC Peptide BEH C18 Column, 300 Å, 1.7 μm, 2.1 mm×100 mm (186003686 Waters Corp.) with a 10 mm guard column of identical packing (186004629 Waters Corp.) coupled to an Impact-II UHR TOF Mass Spectrometer. Liquid chromatography was performed using 0.1% formic acid in water as Solvent A and 0.1% formic acid in acetonitrile as Solvent B at a flow rate of 0.5 mL/min and a 40° C. column temperature. An initial condition of 5% B was held for 1 min before elution of the proteins of interest during a 6.1 min gradient from 5 to 100% B. The column was washed for 2 min at 100% B and equilibrated at 5% B for 2.85 min, giving a total 12 min run time. An MS scan range of 100-3000 m/z with a spectral rate of 8 Hz was used. External calibration was performed. An MS/MS scan was performed to confirm the sequence of the hormones using pseudo multiple reaction monitoring (MRM) MS/MS fragmentation targeted to theoretical peptide masses with the most abundant (+4 and +5) charge states. The targeted ions for the GLP-1 mutant were an m/z of 703.15 and an m/z of 879.19. The targeted ions for GLP-1 were an m/z 671.74 and an m/z of 839.4. Peptides were fragmented using rotating scans with collisional energy of 30 eV and 50 eV and a 0 eV scan to confirm the presence of the unfragmented peptide with a window of ±4 m/z from targeted m/z values. Data were analyzed manually by comparing theoretical b and y ion m/z values calculated with the systemsbiology.net fragment ion calculator to those observed in the experimental data.

### AlphaLISA

AlphaLISA assays were run based on a previous protocol (49) in a 50 mM HEPES pH 7.4, 150 mM NaCl, 1 mg/ml BSA, and 0.015% v/v Triton X-100 buffer (“Alpha buffer”). An Echo 525 acoustic liquid handler was used to dispense materials from a 384-well Polypropylene 2.0 Plus source microplate (Labcyte, PPL-0200) using the 384PP_Plus_GPSA fluid type into a ProxiPlate-384 Plus (Perkin Elmer 6008280) destination plate. Anti-FLAG donor beads (Perkin Elmer) were used to immobilize the peptide hormone and either Protein A (Fc tagged GLP-1 receptor, Sino Biological, 13944-H02H) or Ni-NTA (6× His tagged GH, Acro GHR-H5222 and PTH, Abcam ab182670, receptors) acceptor beads (PerkinElmer) were used to immobilize the extracellular domain of the respective hormone receptors. The final concentrations of the donor and acceptor beads were 0.08 mg/ml and 0.02 mg/ml, respectively. Hormone concentrations from approximately 500 nM to 0 nM and receptor concentrations from 500 nM to 0 nM were cross titrated to observe expected binding patterns and determine the conditions with the highest signal. Alpha buffer, the purified hormone, and the acceptor beads were added initially and allowed to incubate at room temperature for 1 hour. The donor beads were then added and the reaction was allowed to equilibrate for 1 hour at room temperature before reading the results on a Tecan Infinite M1000 Pro using the AlphaLISA filter with an excitation time of 100 ms, an integration time of 300 ms, and a settle time of 20 ms after 10 min of incubation inside the instrument. Prism 9 (GraphPad) was used to plot the data.

### Biolayer Interferometry (BLI)

Hormone binding to the extracellular domain of its receptor was tested by biolayer interferometry (BLI). Ni-NTA sensor tips were incubated in water for a least 10 min and immobilization buffer (TBS-T pH 7.6 with 1 mg/ml BSA, filtered with a 0.2 μm syringe filter (CELLTREAT 229747)) for at least 10 min before use. On an Octet K2 Biolayer Interferometer, his-tagged extracellular domain of the growth hormone receptor (GHR, BioVision 7477-10) was immobilized on the Ni-NTA sensor tip by dipping the tips in a 2.5 μg/ml solution of GHR for 400 s. Binding was assessed with an estimated growth hormone (GH) concentration ranging from 84 nM to 11.5 nM using a 40 s equilibration step (tips in buffer), a 20 s baseline step, 120 s of association (tips in the GH solution), and 900 s of dissociation. No dissociation was observed, so kinetic model curves could not be fit to the data. A commercially produced GH positive control (Tonbo Biosciences 21-7147-U010) ranging from 194 nM to 7.88 nM was also observed to have no dissociation. On a BLITZ Bio-layer interferometer, his-tagged 25 μg/ml GLP-1 receptor (GLP-1R MyBioSource.com MBS949592) and 50 μg/ml parathyroid hormone receptor (PTH, R&D systems 5709-PR-50) were separately immobilized on Ni-NTA tips for at least 5 min. GLP-1 binding was assessed by equilibrating the tips in buffer for 30 s, measuring association by dipping tips in undiluted GLP-1 purified from CFPS reactions as described above and 100 μg/ml of positive control commercially produced GLP-1 (PeproTech 130-08) for 120 s and measuring dissociation for 120 s. PTH binding was assessed by equilibrating tips in buffer for 30 s, measuring association by dipping tips in undiluted PTH produced in CFPS and purified as previously described or in 67 μg/ml of a commercially produced positive control PTH (OriGene SA6052) for 180 s, and measuring dissociation for 120 s.

### Fluorotect SDS-PAGE Gels

Incorporation of a fluorescent lysine residue during in vitro translation reactions was used to test expression of SUMO-hormone constructs. CFPS reactions were run as described above with the addition of 1 μl of FluoroTect™ reagent (Promega L5001) per 50 μl CFPS reaction. After CFPS reaction completion, 3 μl of a 1:50 dilution of RNase A is added to each 50 μl reaction (Omega AC118) and incubated at 37° C. for 10 min. Samples are prepared for SDS-PAGE by adding 5 μl of sample to 4 μl of tricine sample buffer (Bio-Rad 1610739) and 1 μl of 1 M DTT and heat denaturing at 70° C. for 3 min. The samples are run on an 16.5% MINIPROTEAN® Tris-Tricine gel (Bio-Rad 4563066) for 100 V for 90 min at constant voltage. The gels were imaged on a Li-COR Odyssey Fc imager on the 700 nm channel with 30 s of exposure.

### Growth Inhibition Assay

Antimicrobial peptide activity was tested with a plate-based growth inhibition study. A 5 ml culture of Escherichia coli MG1655 was grown overnight on Luria Broth (LB) from a glycerol stock. On the day of the assay, the overnight culture was diluted to an OD of 0.08 in LB and then grown to mid log phase (OD ˜0.5). The mid log phase cells were diluted to 10+ cells/ml (assuming 1 OD=8×10⁸ cells/ml) into LB with 0.005% Antifoam 204 (Sigma A6426) and 1 mg/ml BSA (Sigma-Aldrich A2153-100G). 5 μl of these cells were added to 10 μl of purified antimicrobial peptide in a UV-sterilized 384 well, clear bottom plate (Greiner Bio-One 781096). Positive controls included 10 μl of the wash buffer the hormones were dissolved in and 10 μl of a no DNA control where no DNA template was added to the CFPS reactions, but the sample was treated exactly like the other antimicrobial peptide samples. Negative controls included a final kanamycin concentration of 50 μg/ml and adding 10 μl of the LB with 0.005% Antifoam 204 and 1 mg/ml BSA without E. coli cells. The plate was sealed with a microseal B adhesive (Bio-Rad MSB1001) and incubated in a plate reader with continuous shaking for 18 hr at 37° C. and absorbance measurements at 600 nm every 15 min.

TABLE 1
A summary of the peptide hormones tested in this work(27, 50-64).

| | Abbrevi- | |
|---|---|---|
| Peptide Hormone | ation | Physiological Purpose |
| Insulin Aspart | Ins Asp | Short-acting insulin to lower blood |
| | | glucose |
| Insulin Lispro | Ins Lis | Short-acting insulin |
| Insulin Glargine | Ins Glarg | Long-acting insulin |
| Regular Insulin | Reg Ins | Short-acting insulin |
| Insulin A Chain | InsA | A chain of insulin |
| Insulin B Chain | InsB | B chain of insulin |
| Insulin A Chain | Insulin | A chain of insulin with a leucine |
| Heterodimer | A H | zipper heterodimer |
| Insulin B Chain | Insulin | B chain of insulin with a leucine |
| Heterodimer | B H | zipper heterodimer |
| Oxytocin | Oxy | Stimulates uterine contractions |
| Glucagon | Glcg | Increases blood glucose |
| Glucagon like | GLP-1 | A mutant of GLP-1 meant to mimic |
| peptide-1 mutant | mut | liraglutide |
| Glucagon like | GLP-1 | Stimulates insulin secretion, decreases |
| peptide-1 | | glucagon secretion, slows gastric |
| | | emptying |
| Insulin like growth | IGF-1 | Promotes growth of bones and tissues |
| factor-1 | | |
| Growth hormone | GH | Promotes growth of bones and tissues |
| Leptin | Lept | Regulates hunger to maintain energy |
| | | homeostasis |
| Vasopressin | Vspn | Increases blood pressure, stimulates |
| | | water retention in kidneys |
| Angiotensin II | Ang II | Vasoconstriction to increase blood |
| | | pressure |
| Parathyroid | PTH | Increases serum calcium, stimulates |
| hormone | | bone formation |
| Somatostatin | SST | Inhibits insulin, glucagon, GH, and |
| | | thyroid stimulating hormone secretion |
| Leuprolide | Leu | Gonadotropin releasing hormone that |
| | | can suppress sex steroid hormone |
| | | production with extended administration |

### REFERENCES

1. Yach, D., Hawkes, C., Gould, C. L., and Hofman, K. J. (2004) The global burden of chronic diseases: overcoming impediments to prevention and control, JAMA 291, 2616-2622.
2. Chen, L., Magliano, D. J., and Zimmet, P. Z. (2011) The worldwide epidemiology of type 2 diabetes mellitus-present and future perspectives, Nat Rev Endocrinol 8, 228-236.
3. Kelly, T., Yang, W., Chen, C. S., Reynolds, K., and He, J. (2008) Global burden of obesity in 2005 and projections to 2030, Int J Obes (Lond) 32, 1431-1437.
4. Chooi, Y. C., Ding, C., and Magkos, F. (2019) The epidemiology of obesity, Metabolism 92, 6-10.
5. Clynes, M. A., Harvey, N. C., Curtis, E. M., Fuggle, N. R., Dennison, E. M., and Cooper, C. (2020) The epidemiology of osteoporosis, Br Med Bull 133, 105-117.
6. Meier, J. J. (2012) GLP-1 receptor agonists for individualized treatment of type 2 diabetes mellitus, Nat Rev Endocrinol 8, 728-742.
7. Burcelin, R., and Gourdy, P. (2017) Harnessing glucagon-like peptide-1 receptor agonists for the pharmacological treatment of overweight and obesity, Obes Rev 18, 86-98.
8. Wilding, J. P. H., Batterham, R. L., Calanna, S., Davies, M., Van Gaal, L. F., Lingvay, I., McGowan, B. M., Rosenstock, J., Tran, M. T. D., Wadden, T. A., Wharton, S., Yokote, K., Zeuthen, N., Kushner, R. F., and Group, S. S. (2021) Once-Weekly Semaglutide in Adults with Overweight or Obesity, N Engl J Med 384, 989-1002.
9. Diez, J. J., Sangiao-Alvarellos, S., and Cordido, F. (2018) Treatment with Growth Hormone for Adults with Growth Hormone Deficiency Syndrome: Benefits and Risks, Int J Mol Sci 19.
10. Haffner, D., Schaefer, F., Nissel, R., Wuhl, E., Tonshoff, B., and Mehls, O. (2000) Effect of growth hormone treatment on the adult height of children with chronic renal failure. German Study Group for Growth Hormone Treatment in Chronic Renal Failure, N Engl J Med 343, 923-930.
11. Tamhane, S., Sfeir, J. G., Kittah, N. E. N., Jasim, S., Chemaitilly, W., Cohen, L. E., and Murad, M. H. (2018) GH Therapy in Childhood Cancer Survivors: A Systematic Review and Meta-Analysis, J Clin Endocrinol Metab 103, 2794-2801.
12. Crandall, C. (2002) Parathyroid hormone for treatment of osteoporosis, Arch Intern Med 162, 2297-2309.
13. Rejnmark, L., Underbjerg, L., and Sikjaer, T. (2015) Hypoparathyroidism: Replacement Therapy with Parathyroid Hormone, Endocrinol Metab (Seoul) 30, 436-442.
14. Yu, Y. B., Briggs, K. T., Taraban, M. B., Brinson, R. G., and Marino, J. P. (2021) Grand Challenges in Pharmaceutical Research Series: Ridding the Cold Chain for Biologics, Pharm Res 38, 3-7.
15. Brechtelsbauer, E., and Shah, S. (2020) Update on pharmaceutical waste disposal regulations: Strategies for success, Am J Health Syst Pharm 77, 574-582.
16. Carlson, E. D., Gan, R., Hodgman, C. E., and Jewett, M. C. (2012) Cell-free protein synthesis: applications come of age, Biotechnol Adv 30, 1185-1194.
17. Warfel, K. F., Williams, A., Wong, D. A., Sobol, S. E., Desai, P., Li, J., Chang, Y.-F., DeLisa, M. P., Karim, A. S., and Jewett, M. C. (2022) A low-cost, thermostable, cell-free protein synthesis platform for on demand production of conjugate vaccines, bioRxiv, 2022.2008.2010.503507.
18. Stark, J. C., Jaroentomeechai, T., Moeller, T. D., Hershewe, J. M., Warfel, K. F., Moricz, B. S., Martini, A. M., Dubner, R. S., Hsu, K. J., Stevenson, T. C., Jones, B. D., DeLisa, M. P., and Jewett, M. C. (2021) On-demand biomanufacturing of protective conjugate vaccines, Sci Adv 7.
19. Pardee, K., Slomovic, S., Nguyen, P. Q., Lee, J. W., Donghia, N., Burrill, D., Ferrante, T., McSorley, F. R., Furuta, Y., Vernet, A., Lewandowski, M., Boddy, C. N., Joshi, N. S., and Collins, J. J. (2016) Portable, On-Demand Biomolecular Manufacturing, Cell 167, 248-259 e212.
20. Vaca Gonzalez, C. P., Arteaga, L., and Delgado Lopez, N. E. (2019) Magistral drug production in Colombia and other middle-income countries, Nat Biotechnol 37, 216-217.
21. Adiga, R., Al-Adhami, M., Andar, A., Borhani, S., Brown, S., Burgenson, D., Cooper, M. A., Deldari, S., Frey, D. D., Ge, X., Guo, H., Gurramkonda, C., Jensen, P., Kostov, Y., LaCourse, W., Liu, Y., Moreira, A., Mupparapu, K., Penalber-Johnstone, C., Pilli, M., Punshon-Smith, B., Rao, A., Rao, G., Rauniyar, P., Snovida, S., Taurani, K., Tilahun, D., Tolosa, L., Tolosa, M., Tran, K., Vattem, K., Veeraraghavan, S., Wagner, B., Wilhide, J., Wood, D. W., and Zuber, A. (2018) Point-of-care production of therapeutic proteins of good-manufacturing-practice quality, Nat Biomed Eng 2, 675-686.
22. Algorri, M., Abernathy, M. J., Cauchon, N. S., Christian, T. R., Lamm, C. F., and Moore, C. M. V. (2022) Re-Envisioning Pharmaceutical Manufacturing: Increasing Agility for Global Patient Access, J Pharm Sci 111, 593-607.
23. Kalkman, S., Arentshorst, M., Hoekman, J., Boon, W., Uijtendaal, E., van Thiel, G., and Moors, E. (2020) In-Hospital Production of Medicines: Preparing for Disruption, Trends Biotechnol 38, 1045-1047.
24. Fankhauser, D., Alissandratos, A., Liutkus, M., and Easton, C. J. (2021) Easy Production of “Difficult Peptides” Using Cell-Free Protein Synthesis and a New Methionine Analogue as a Latent Peptide Cleavage Site, Chemistry 27, 17487-17494.
25. Ryan, B. J., and Henehan, G. T. (2013) Overview of approaches to preventing and avoiding proteolysis during expression and purification of proteins, Curr Protoc Protein Sci Chapter 5, Unit 5 25.
26. Zorko, M., and Jerala, R. (2010) Production of recombinant antimicrobial peptides in bacteria, Methods Mol Biol 618, 61-76.
27. Ojima-Kato, T., Morishita, S., Uchida, Y., Nagai, S., Kojima, T., and Nakano, H. (2018) Rapid Generation of Monoclonal Antibodies from Single B Cells by Ecobody Technology, Antibodies (Basel) 7.
28. Marblestone, J. G., Edavettal, S. C., Lim, Y., Lim, P., Zuo, X., and Butt, T. R. (2006) Comparison of SUMO fusion technology with traditional gene fusion systems: enhanced expression and solubility with SUMO, Protein Sci 15, 182-189.
29. Terpe, K. (2003) Overview of tag protein fusions: from molecular and biochemical fundamentals to commercial systems, Appl Microbiol Biotechnol 60, 523-533.
30. Peroutka Iii, R. J., Orcutt, S. J., Strickler, J. E., and Butt, T. R. (2011) SUMO Fusion Technology for Enhanced Protein Expression and Purification in Prokaryotes and Eukaryotes, Methods in Molecular Biology 705, 15-30.
31. Celen, A. B., and Sahin, U. (2020) Sumoylation on its 25th anniversary: mechanisms, pathology, and emerging concepts, FEBS J 287, 3110-3140.
32. Malakhov, M. P., Mattern, M. R., Malakhova, O. A., Drinker, M., Weeks, S. D., and Butt, T. R. (2004) SUMO fusions and SUMO-specific protease for efficient expression and purification of proteins, J Struct Funct Genomics 5, 75-86.
33. Feldman, M. F., Wacker, M., Hernandez, M., Hitchen, P. G., Marolda, C. L., Kowarik, M., Morris, H. R., Dell, A., Valvano, M. A., and Aebi, M. (2005) Engineering N-linked protein glycosylation with diverse O antigen lipopolysaccharide structures in Escherichia coli, Proc Natl Acad Sci USA 102, 3016-3021.
34. Des Soye, B. J., Gerbasi, V. R., Thomas, P. M., Kelleher, N. L., and Jewett, M. C. (2019) A Highly Productive, One-Pot Cell-Free Protein Synthesis Platform Based on Genomically Recoded Escherichia coli, Cell Chem Biol 26, 1743-1754 e1749.
35. Kielgast, U., Holst, J. J., and Madsbad, S. (2011) Antidiabetic actions of endogenous and exogenous GLP-1 in type 1 diabetic patients with and without residual beta-cell function, Diabetes 60, 1599-1607.
36. Wettergren, A., Pridal, L., Wojdemann, M., and Holst, J. J. (1998) Amidated and non-amidated glucagon-like peptide-1 (GLP-1): non-pancreatic effects (cephalic phase acid secretion) and stability in plasma in humans, Regul Pept 77, 83-87.
37. Son, J. M., Ahn, J. H., Hwang, M. Y., Park, C. G., Choi, C. Y., and Kim, D. M. (2006) Enhancing the efficiency of cell-free protein synthesis through the polymerase-chain-reaction-based addition of a translation enhancer sequence and the in situ removal of the extra amino acid residues, Anal Biochem 351, 187-192.
38. Zhu, K., Zhou, X., Yan, Y., Mo, H., Xie, Y., Cheng, B., and Fan, J. (2017) Cleavage of fusion proteins on the affinity resins using the TEV protease variant, Protein Expr Purif 131, 27-33.
39. Walker, P. A., Leong, L. E., Ng, P. W., Tan, S. H., Waller, S., Murphy, D., and Porter, A. G. (1994) Efficient and rapid affinity purification of proteins using recombinant fusion proteases, Biotechnology (N Y) 12, 601-605.
40. Dian, C., Eshaghi, S., Urbig, T., McSweeney, S., Heijbel, A., Salbert, G., and Birse, D. (2002) Strategies for the purification and on-column cleavage of glutathione-S-transferase fusion target proteins, J Chromatogr B Analyt Technol Biomed Life Sci 769, 133-144.
41. Frey, S., and Gorlich, D. (2014) Purification of protein complexes of defined subunit stoichiometry using a set of orthogonal, tag-cleaving proteases, J Chromatogr A 1337, 106-115.
42. Zawada, J. F., Yin, G., Steiner, A. R., Yang, J., Naresh, A., Roy, S. M., Gold, D. S., Heinsohn, H. G., and Murray, C. J. (2011) Microscale to manufacturing scale-up of cell-free cytokine production—a new approach for shortening protein production development timelines, Biotechnol Bioeng 108, 1570-1578.
43. Peterson, G. E., and Pollom, R. D. (2010) Liraglutide in clinical practice: dosing, safety and efficacy, Int J Clin Pract Suppl, 35-43.
44. Hodsman, A. B., Hanley, D. A., Ettinger, M. P., Bolognese, M. A., Fox, J., Metcalfe, A. J., and Lindsay, R. (2003) Efficacy and safety of human parathyroid hormone-(1-84) in increasing bone mineral density in postmenopausal osteoporosis, J Clin Endocrinol Metab 88, 5212-5220.
45. Beaudet, L., Rodriguez-Suarez, R., Venne, M.-H., Caron, M., Bédard, J., Brechler, V., Parent, S., and Bielefeld-Sévigny, M. (2008) AlphaLISA immunoassays: the no-wash alternative to ELISAs for research and drug discovery, Nature Methods 5, an8-an9.
46. Layton, C. J., McMahon, P. L., and Greenleaf, W. J. (2019) Large-Scale, Quantitative Protein Assays on a High-Throughput DNA Sequencing Chip, Mol Cell 73, 1075-1082 e1074
47. Jewett, M. C., and Swartz, J. R. (2004) Mimicking the Escherichia coli cytoplasmic environment activates long-lived and efficient cell-free protein synthesis, Biotechnol Bioeng 86, 19-26.
48. Kim, D. M., Kigawa, T., Choi, C. Y., and Yokoyama, S. (1996) A highly efficient cell-free protein synthesis system from Escherichia coli, Eur J Biochem 239, 881-886.
49. Hunt, A. C., Case, J. B., Park, Y. J., Cao, L., Wu, K., Walls, A. C., Liu, Z., Bowen, J. E., Yeh, H. W., Saini, S., Helms, L., Zhao, Y. T., Hsiang, T. Y., Starr, T. N., Goreshnik, I., Kozodoy, L., Carter, L., Ravichandran, R., Green, L. B., Matochko, W. L., Thomson, C. A., Vogeli, B., Kruger, A., VanBlargan, L. A., Chen, R. E., Ying, B., Bailey, A. L., Kafai, N. M., Boyken, S. E., Ljubetic, A., Edman, N., Ueda, G., Chow, C. M., Johnson, M., Addetia, A., Navarro, M. J., Panpradist, N., Gale, M., Jr., Freedman, B. S., Bloom, J. D., Ruohola-Baker, H., Whelan, S. P. J., Stewart, L., Diamond, M. S., Veesler, D., Jewett, M. C., and Baker, D. (2022) Multivalent designed proteins neutralize SARS-CoV-2 variants of concern and confer protection against infection in mice, Sci Transl Med 14, eabn 1252.
50. Hermansen, K., Fontaine, P., Kukolja, K. K., Peterkova, V., Leth, G., and Gall, M. A. (2004) Insulin analogues (insulin detemir and insulin aspart) versus traditional human insulins (NPH insulin and regular human insulin) in basal-bolus therapy for patients with type 1 diabetes, Diabetologia 47, 622-629.
51. Zinman, B., Tildesley, H., Chiasson, J. L., Tsui, E., and Strack, T. (1997) Insulin lispro in CSII: results of a double-blind crossover study, Diabetes 46, 440-443.
52. Bolli, G. B., and Owens, D. R. (2000) Insulin glargine, Lancet 356, 443-445.
53. Mayer, J. P., Zhang, F., and DiMarchi, R. D. (2007) Insulin structure and function, Biopolymers 88, 687-713.
54. Blanks, A. M., and Thornton, S. (2003) The role of oxytocin in parturition, BJOG 110 Suppl 20, 46-51.
55. Jiang, G., and Zhang, B. B. (2003) Glucagon and regulation of glucose metabolism, Am J Physiol Endocrinol Metab 284, E671-678.
56. Nadkarni, P., Chepurny, O. G., and Holz, G. G. (2014) Chapter Two-Regulation of Glucose Homeostasis by GLP-1, In Progress in Molecular Biology and Translational Science (Tao, Y.-X., Ed.), pp 23-65, Academic Press.
57. Laron, Z. (2001) Insulin-like growth factor 1 (IGF-1): a growth hormone, Mol Pathol 54, 311-316.
58. Strobl, J. S., and Thomas, M. J. (1994) Human growth hormone, Pharmacol Rev 46, 1-34.
59. Friedman, J. (2014) 20 years of leptin: leptin at 20: an overview, J Endocrinol 223, T1-8.
60. Holt, N. F., and Haspel, K. L. (2010) Vasopressin: a review of therapeutic applications, J Cardiothorac Vase Anesth 24, 330-347.
61. Tumlin, J. A., Murugan, R., Deane, A. M., Ostermann, M., Busse, L. W., Ham, K. R., Kashani, K., Szerlip, H. M., Prowle, J. R., Bihorac, A., Finkel, K. W., Zarbock, A., Forni, L. G., Lynch, S. J., Jensen, J., Kroll, S., Chawla, L. S., Tidmarsh, G. F., Bellomo, R., and Angiotensin, I. I. f. t. T. o. H.-O. S. I. (2018) Outcomes in Patients with Vasodilatory Shock and Renal Replacement Therapy Treated with Intravenous Angiotensin II, Crit Care Med 46, 949-957.
62. Greenspan, S. L., Bone, H. G., Ettinger, M. P., Hanley, D. A., Lindsay, R., Zanchetta, J. R., Blosch, C. M., Mathisen, A. L., Morris, S. A., Marriott, T. B., and Treatment of Osteoporosis with Parathyroid Hormone Study, G. (2007) Effect of recombinant human parathyroid hormone (1-84) on vertebral fracture and bone mineral density in postmenopausal women with osteoporosis: a randomized trial, Ann Intern Med 146, 326-339.
63. Harris, A. G. (1994) Somatostatin and somatostatin analogues: pharmacokinetics and pharmacodynamic effects, Gut 35, S1-4.
64. Wilson, A. C., Meethal, S. V., Bowen, R. L., and Atwood, C. S. (2007) Leuprolide acetate: a drug of diverse clinical applications, Expert Opin Investig Drugs 16, 1851-1863.
U.S. Pat. No. 5,424,199A—Human Growth Hormone
U.S. Pat. No. 4,086,196A—Parathyroid Hormone
WO2014199397A2—Process for the preparation of liraglutide
Bancroft, F. C., Wu, G., & Zubay, G. (1973). Cell-Free Synthesis of Rat Growth Hormone. Proc. Nat. Acad. Sci., 70 (12), 3646-3649.
Hu, Y., Zhang, Y., Xu, Y., & Liu, Tx. (2017). Synthesis of soluble proinsulin in a cell-free protein synthesis system. Chinese Journal of Biology. 33 (3), 367-377. Doi: 13345/j.cjb.160452
Johnson, D. E., Bauer, G. E., Williams, F. G., & Dixit, P. K. (1984). The synthesis of insulin and proinsulin in a cell-free system derived from the streptozotocinnicotinamide-induced rat islet adenoma. Metabolism. 33 (1) 8-14. doi: 10.1016/0026-0495 (84) 90155-0.
Wen, C., Gan, R., & Zhu, S. (2003). Construction of a secretory expression system suitable to express glucagon under the control of PL promoter. Current Microbiology. 47 180-185. doi: 10/1007/s00284-002-3988-y.
Kommer, A. A., Dashkova, I. G., Esipov, R. S., Miroshnikov, A. I., & Spirin, A. S. (2005). Synthesis of functionally active human proinsulin in a cell-free translation system. Biochemistry, Biophysics, and Molecular Biology, 401 (5), 696-700. Retrieved from https://link.springer.com/content/pdf/10.1007/s10628-005-0058-y.pd
Timm, A. C., Shankles, P. G., Foster, C. M., Doktycz, M. J., & Retterer, S. T. (2016). Toward Microfluidic Reactors for Cell-Free Protein Synthesis at the Point-of-Care. Small, 12 (6), 810-817. https://doi.org/10.1002/smll.201502764
Adiga, R., Al-adhami, M., Andar, A., Borhani, S., Brown, S., Burgenson, D., . . . Zuber, A. (2018). Point-of-care production of therapeutic proteins of good-manufacturing-practice quality. Nature Biomedical Engineering 2018 2:9, 2 (9), 675-686. https://doi.org/10.1038/s41551-018-0259-1
Ogonah, O. W., Polizzi, K. M., & Bracewell, D. G. (2017). Cell free protein synthesis: a viable option for stratified medicines manufacturing? Current Opinion in Chemical Engineering, 18, 77-83. https://doi.org/10.1016/J.COCHE.2017.10.003
Stark, J. C., Jaroentomeechai, T., Moeller, T. D., Hershewe, J. M., Warfel, K. F., Moricz, B. S., . . . Jewett, M. C. (2021). On-demand biomanufacturing of protective conjugate vaccines. Science Advances, 7 (6), eabe9444. https://doi.org/10.1126/sciadv.abe9444
Zhou, L., Wang, H. Y., Tong, S., Okamoto, C. T., Shen, W. C., & Zaro, J. L. (2017). Single chain Fc-dimer-human growth hormone fusion protein for improved drug delivery. Biomaterials, 117, 24-31. https://doi.org/10.1016/J.BIOMATERIALS.2016.11.051
Amet, N., Wang, W., & Shen, W. C. (2010). Human growth hormone-transferrin fusion protein for oral delivery in hypophysectomized rats. Journal of Controlled Release, 141 (2), 177-182. https://doi.org/10.1016/J.JCONREL.2009.09.007
Gardellal, T. J., Rubin, D., Abou-Samra, A.-B., Keutmann, H. T., Potts, J. T., Kronenberg, H. M., & Nussbaum, S. R. (1990). Expression of Human Parathyroid Hormone-(1-84) in Escherichia coli as a Factor X-cleavable Fusion Protein*. The Journal of Biological Chemistry, 265 (26), 15854-15859. https://doi.org/10.1016/S0021-9258(18)55477-6
Astrid B. Jensen, Franta Hubálek, Carsten Enggaard Stidsen, Eva Johansson, Fredrik Kryh Öberg, Michael Skjøt, Thomas Kjeldsen, Cell free protein synthesis versus yeast expression—A comparison using insulin as a model protein, Protein Expression and Purification, Volume 186, 2021. https://www.sciencedirect.com/science/article/pii/S1046592821000930
Keith Pardee, Shimyn Slomovic, Peter Q. Nguyen, Jeong Wook Lee, Nina Donghia, Devin Burrill, Tom Ferrante, Fern R. McSorley, Yoshikazu Furuta, Andyna Vernet, Michael Lewandowski, Christopher N. Boddy, Neel S. Joshi, James J. Collins, Portable, On-Demand Biomolecular Manufacturing, Cell, Volume 167, Issue 1. 2016. https://www.sciencedirect.com/science/article/pii/S0092867416312466
Astrid B. Jensen, Franta Hubálek, Carsten Enggaard Stidsen, Eva Johansson, Fredrik Kryh Öberg, Michael Skjøt, Thomas Kjeldsen, Cell free protein synthesis versus yeast expression—A comparison using insulin as a model protein, Protein Expression and Purification, Volume 186, 2021 https://www.sciencedirect.com/science/article/pii/S1046592821000930
Mark Thomas Smith, Scott D. Berkheimer, Christopher J. Werner, and Bradley C. Bundy. Lyophilized Escherichia coli-based cell-free systems for robust, high-density, long-term storage. BioTechniques 2014 56:4, 186-193. https://www.futurescience.com/doi/10.2144/000114158
K. A. Martemyanov, V. A. Shirokov, O. V. Kurnasov, A. T. Gudkov, A. S. Spirin Cell-free production of biologically active polypeptides: application to the synthesis of antibacterial peptide cecropin Protein Expr. Purif., 21 (2001), pp. 456-461 https://www.sciencedirect.com/science/article/pii/S1046592801914007

Ribosomal synthesis and in situ isolation of peptide molecules in a cell-free translation system. Protein Expr. Purif., 71 (2010), pp. 16-20. https://www.sciencedirect.com/science/article/pii/S1046592810000264

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

All methods described herein can be performed in any suitable order unless otherwise indicated herein or otherwise clearly contradicted by context. The use of any and all examples provided herein, is intended merely to better illuminate the invention and does not pose a limitation on the scope of the invention unless otherwise claimed. No language in the specification should be construed as indicating any non-claimed element as essential to the practice of the invention.

Citations to a number of patent and non-patent references are made herein. The cited references are incorporated by reference herein in their entireties. In the event that there is an inconsistency between a definition of a term in the specification as compared to a definition of the term in a cited reference, the term should be interpreted based on the definition in the specification.

It will be understood by one of ordinary skill in the art that reaction components are routinely stored as separate solutions, each containing a subset of the total components, for reasons of convenience, storage stability, or to allow for application-dependent adjustment of the component concentrations, and that reaction components are combined prior to the reaction to create a complete reaction mixture. Furthermore, it will be understood by one of ordinary skill in the art that reaction components are packaged separately for commercialization and that useful commercial kits may contain any subset of the reaction components of the invention.

The methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A nucleic acid construct encoding a fusion protein comprising:
a therapeutic protein and one or more of:
(a) a CAT enhancer;
(b) a purification tag;
(c) a flexible linker;
(d) an internal fusion protein (IFP).

2. The construct of claim 1, comprising double-stranded DNA.

3. The construct of claim 2, comprising a vector.

4. The construct of claim 3, wherein the vector comprises an expression vector.

5. The construct of claim 1, comprising RNA.

6. The construct of claim 1, wherein the therapeutic protein comprises one or more of insulin aspartate (InsAsp), insulin lispro (InsLis), insulin glargine (INsGlar), insulin (Reg Ins), insulin A chain (InsA), insulin B chain (InsB), insulin A chain with N-terminal heterodimer (InsA H), Insulin B chain with N-terminal heterodimer (InsB H), oxytocin (Oxy), glucagon (Gcg), liraglutide (Lira), glucagon-like peptide-1 (GLP-1), glucagon like peptide-1 mutant (GLP-1 mut), insulin-like growth factor-1 (IGF-1), growth hormone (GH), leptin (Lept), vasopressin (Vspn), angiotensin II (Ang II), parathyroid hormone (PTH), somatostatin (SST), leuprolide (Leu).

7. The construct of claim 1, wherein (d) comprises a cleavable moiety, and optionally, wherein (d) comprises a solubility/stability enhancing protein.

8. The construct of claim 1, wherein (d) comprises a small ubiquitin-related modifier (SUMO).

9. A method of producing a therapeutic protein, the method comprising:
(a) contacting the nucleic acid construct of claim 1 to a cell-free protein synthesis (CFPS) system under conditions for the transcription and translation of the nucleic acid construct to produce the encoded fusion protein;
(b) contacting the fusion protein to a capture matrix, wherein the purification tag of the fusion protein binds to the capturing matrix;
(c) eluting the therapeutic protein from the capture matrix.

10. The method of claim 9, wherein eluting the therapeutic protein from the capture matrix comprises cleaving the IFP of the fusion protein, wherein the IFP comprises a cleavage site.

11. A method of producing a therapeutic protein, the method comprising:
(a) contacting a nucleic acid construct of claim 5 to a cell-free protein synthesis (CFPS) system under conditions for the translation of the nucleic acid construct to produce the encoded fusion protein;
(b) contacting the fusion protein to a capture matrix, wherein the purification tag of the fusion protein binds to the capturing matrix;
(c) eluting the therapeutic protein from the capture matrix.

12. The method of claim 11, wherein eluting the therapeutic protein from the capture matrix comprises cleaving the IFP of the fusion protein, wherein the IFP comprises a cleavage site.

13. The method 9, wherein the CFPS system comprise an extract that facilitates disulfide bond formation.

14. The method of claim 9, wherein the CFPS system comprise an extract that facilitates glycosylation of the fusion protein.

15. The method of claim 9, wherein the CFPS system is lyophilized, and wherein the method comprises rehydration of the CFPS system.

16. The construct of claim 1, comprising:
a cAT enhancer,
a purification tag selected from Strep-II, Flag, Fc, or His;
optionally, a flexible linker;
a small ubiquitin-related modifier (SUMO);
wherein the therapeutic protein is one of: insulin aspartate (InsAsp), insulin lispro (InsLis), insulin glargine (INsGlar), insulin (Reg Ins), insulin A chain (InsA), insulin B chain (InsB), insulin A chain with N-terminal heterodimer (InsA H), Insulin B chain with N-terminal heterodimer (InsB H), oxytocin (Oxy), glucagon (Gcg), liraglutide (Lira), glucagon-like peptide-1 (GLP-1), glucagon like peptide-1 mutant (GLP-1 mut), insulin-like growth factor-1 (IGF-1), growth hormone (GH), leptin (Lept), vasopressin (Vspn), angiotensin II (Ang II), parathyroid hormone (PTH), somatostatin (SST), leuprolide (Leu).


SEQUENCE LISTING (33 sequences)

SEQ ID NO: 1 - protein, 5 aa, synthetic construct
MEKKI

SEQ ID NO: 2 - protein, 10 aa, synthetic construct
WSHPQFEKTG

SEQ ID NO: 3 - protein, 7 aa, synthetic construct
GGGSGGG

SEQ ID NO: 4 - protein, 8 aa, synthetic construct
DYKDEDLL

SEQ ID NO: 5 - protein, 5 aa, synthetic construct
GSSGS

SEQ ID NO: 6 - protein, 101 aa, synthetic construct
MASGSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGG

SEQ ID NO: 7 - protein, 86 aa, synthetic construct
FVNQHLCGSHLVEALYLVCGERGFFYTDKTRREAEDLQVGQVELGGGPGAGSLQPLALEGSLQKRGIVEQCCTSICSLYQLENYCN

SEQ ID NO: 8 - protein, 86 aa, synthetic construct
FVNQHLCGSHLVEALYLVCGERGFFYTKPTRREAEDLQVGQVELGGGPGAGSLQPLALEGSLQKRGIVEQCCTSICSLYQLENYCN

SEQ ID NO: 9 - protein, 86 aa, synthetic construct
FVNQHLCGSHLVEALYLVCGERGFFYTPKTRREAEDLQVGQVELGGGPGAGSLQPLALEGSLQKRGIVEQCCTSICSLYQLENYCG

SEQ ID NO: 10 - protein, 86 aa, synthetic construct
FVNQHLCGSHLVEALYLVCGERGFFYTPKTRREAEDLQVGQVELGGGPGAGSLQPLALEGSLQKRGIVEQCCTSICSLYQLENYCN

SEQ ID NO: 11 - protein, 21 aa, synthetic construct
GIVEQCCTSICSLYQLENYCN

SEQ ID NO: 12 - protein, 30 aa, synthetic construct
FVNQHLCGSHLVEALYLVCGERGFFYTPKT

SEQ ID NO: 13 - protein, 77 aa, synthetic construct
GTKEDILERQRKIIERAQEIHRRQQEILEELERIIRKPGSSEEAMKRMLKLLEESLRLLKELLELSEESAQLLYEQR

SEQ ID NO: 14 - protein, 83 aa, synthetic construct
GTEKRLLEEAERAHREQKEIIKKAQELHRRLEEIVRQSGSSEEAKKEAKKILEEIRELSKRSLELLREILYLSQEQKGSLVPR

SEQ ID NO: 15 - protein, 9 aa, synthetic construct
CYIQNCPLG

SEQ ID NO: 16 - protein, 29 aa, synthetic construct
HSQGTFTSDYSKYLDSRRAQDFVQWLMNT

SEQ ID NO: 17 - protein, 32 aa, synthetic construct
HAEGTFTSDVSSYLEGQAAKEEFIAWLVRGRG

SEQ ID NO: 18 - protein, 31 aa, synthetic construct
HAEGTFTSDVSSYLEGQAAKEFIAWLVKGRG

SEQ ID NO: 19 - protein, 70 aa, synthetic construct
GPETLCGAELVDALQFVCGDRGFYFNKPTGYGSSSRRAPQTGIVDECCFRSCDLRRLEMYCAPLKPAKSA

SEQ ID NO: 20 - protein, 191 aa, synthetic construct [sequence omitted]

SEQ ID NO: 21 - protein, 146 aa, synthetic construct
VPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGC

SEQ ID NO: 22 - protein, 9 aa, synthetic construct
CYFQNCPRG

SEQ ID NO: 23 - protein, 8 aa, synthetic construct
DRVYIHPF

SEQ ID NO: 24 - protein, 84 aa, synthetic construct
SVSEIQLMHNLGKHLNSMERVEWLRKKLQDVHNFVALGAPLAPRDAGSQRPRKKEDNVLVESHEKSLGEADKADVNVLTKAKSQ

SEQ ID NO: 25 - protein, 28 aa, synthetic construct
SANSNPAMAPRERKAGCKNFFWKTFTSC

SEQ ID NO: 26 - protein, 10 aa, synthetic construct
QHWSYGLRPG

SEQ ID NO: 27 - protein, 31 aa, synthetic construct
TKLTEEEKNRLNFLKKISQRYQKFALPQYLK

SEQ ID NO: 28 - protein, 35 aa, synthetic construct
KWKIFKKIEKVGRNIRNGIIKAGPAVAVLGEAKAL

SEQ ID NO: 29 - protein, 26 aa, synthetic construct
GIGAVLKVLTTGLPALISWIKRKRQQ

SEQ ID NO: 30 - protein, 37 aa, synthetic construct
WNPFKELERAGQRVRDAVISAAPAVATVGQAAAIARG

SEQ ID NO: 31 - protein, 21 aa, synthetic construct
SSMKLSFRARAYGFRGPGPQL

SEQ ID NO: 32 - protein, 68 aa, synthetic construct
AELRCMCIKTTSGIHPKNIQSLEVIGKGTHCNQVEVIATLKKGRKICLDPDAPRIKKIVQKKLAGDES

SEQ ID NO: 33 - protein, 68 aa, synthetic construct
AELRCMCIKTTSGIHPKNIQSLEVIGKGTHCNQVEVIATLKDGRKICLDPDAPRIKKIVQKKLAGDES